SEQUENCE LISTING (1 sequence)

SEQ ID NO: 1 - protein, 27 aa, Homo Sapiens
FITCACADLDLEALAPYIPADDDFQLR

(19) United States
(12) Patent Application Publication
(10) Pub. No.: US 2011/0028507 A1
(43) Pub. Date: Feb. 3, 2011

(54) PYRIDINE DERIVATIVES AND METHODS OF USE THEREOF

(75) Inventors: Sun Nam Kim, Seoul (KR); Yong Rae Hong, Seoul (KR); Dongkyu Shin, Seoul (KR); Seonggu Ro, Seoul (KR); Joong Myung Cho, Seoul (KR); Mi Jung Lee, Seoul (KR); Ho Jin Chang, Seoul (KR)

Correspondence Address: WOODCOCK WASHBURN LLP, CIRA CENTRE, 12TH FLOOR, 2929 ARCH STREET, PHILADELPHIA, PA, 19104-2891, US

(73) Assignee: CRYSTALGENOMICS, INC., Seoul (KR)

(21) Appl. No.: 12/672,785

(22) PCT Filed: Aug. 8, 2008
(86) PCT No.: PCT/IB2008/003144

§ 371 Date: Oct. 8, 2010

Related U.S. Application Data
(60) Provisional application No. 60/955,193, filed on Aug. 10, 2007.

(51) Int. Cl. A61K 31/4365; C07D 513/04; C12N 5/00; C12N 9/99; C07D 491/04; A61K 31/4355; A61P 9/10; A61P 7/06; A61P 19/02; A61P 29/00; A61P 1/00; A61P 25/00; A61P 25/08; A61P 31/18; A61P 13/12; A61P 3/00; A61P 37/06

(52) U.S. Cl. 514/291; 546/80; 435/375; 435/184; 546/115; 514/302

(57) ABSTRACT

Disclosed herein are pyridine derivatives, or a pharmaceutically acceptable salt, ester, amide, or prodrug thereof, pharmaceutical compositions comprising the same, and methods of modulating the level or activity of HIF in a subject, inhibiting hydroxylation of HIF α in a subject, modulating expression of HIF-regulated genes in a subject, treating an HIF-related disorder in a subject, increasing levels of endogenous EPO in a subject, or treating a disorder in a subject, using the disclosed compounds.

### RELATED APPLICATIONS

The present application claims priority to the U.S. Provisional Application Ser. No. 60/955,193, filed on Aug. 10, 2007, the entire disclosure of which is incorporated by reference herein.

### FIELD OF THE INVENTION

The present invention is in the field of pharmaceutical chemistry, and particularly in the field of compounds that affect the stability of hypoxia inducible factor-α(HIF-α) and the expression of HIF-regulated genes, and methods of using the same for the treatment of disease.

### BACKGROUND OF THE DISCLOSURE

The hypoxia-inducible factor (HIF) family of transcription factors play a central regulatory role in the control of the intracellular response to hypoxia, throughout the body. HIF itself is primarily regulated by prolyl hydroxylases (PHDs), as well as asparaginyl hydroxylases. Under normoxic conditions, these PHDs site specifically hydroxylate the alpha subunit of HIF, which ultimately results in its degradation. Thus, under adequate oxygenation levels, the body continually expresses and degrades the HIF alpha protein.

Modulation of PHD via the compounds disclosed herein, will alter the regulation of cellular oxygen homeostasis. This has utility in any disease state where ischemia, hypoxia, and/or anemia plays a role

### SUMMARY OF THE INVENTION

Disclosed herein are compounds selected from the group consisting of Formula I, Formula II, Formula III, Formula IV, and Formula V:

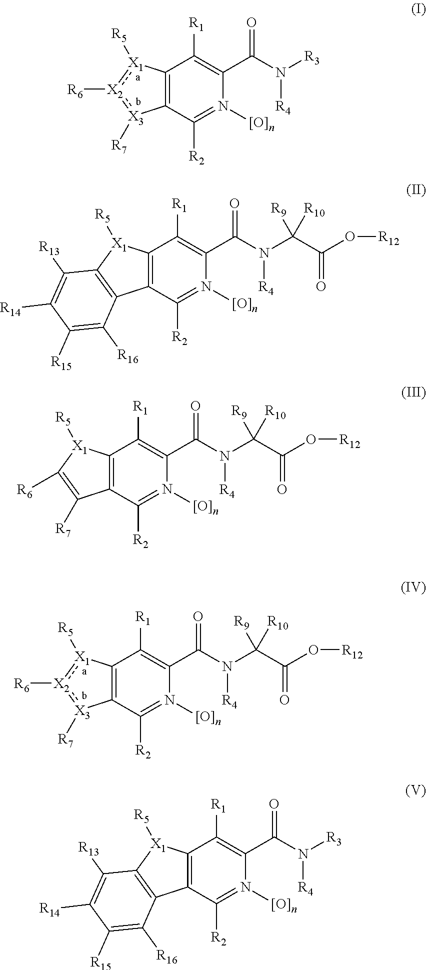

or a pharmaceutically acceptable salt, ester, amide, or prodrug thereof,
wherein
n is 0 or 1;
R₁ is —OR₈ or halo;
R₂ is selected from the group consisting of hydrogen, optionally substituted alkyl, optionally substituted cycloalkyl, optionally substituted aryl, halo, and cyano;
R₃ is selected from the group consisting of optionally substituted cycloalkyl, optionally substituted aryl, optionally substituted heteroaryl, —CR₉R₁₀R₁₁, and —CR₉R₁₀—C(═O)OR₁₂;
R₄ is hydrogen or —OR₈;
X₁ is selected from the group consisting of oxygen, sulfur, and carbon;
R₅ is selected from the group consisting of hydrogen, optionally substituted alkyl, optionally substituted cycloalkyl, optionally substituted aryl, and optionally substituted heteroaryl,
   provided that R₅ does not exist when X₁ is oxygen or sulfur;
X₂ and X₃ are each independently nitrogen or carbon,
   provided that at least one of X₁, X₂, and X₃ is carbon;
R₆ is selected from the group consisting of hydrogen, optionally substituted alkyl, optionally substituted cycloalkyl, optionally substituted aryl, optionally substituted heteroaryl, and OR₈;
R₇ is selected from the group consisting of hydrogen, optionally substituted alkyl, optionally substituted cycloalkyl, optionally substituted aryl, optionally substituted heteroaryl, and —SO₂—Ar, wherein Ar is an optionally substituted aryl; or
X₂ and X₃ are both carbon and R₆ and R₇ taken together along with the carbon atoms to which they are attached form a ring of formula

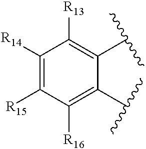

wherein
R₁₃ and R₁₄ are each independently selected from the group consisting of hydrogen, optionally substituted alkyl, optionally substituted cycloalkyl, optionally substituted aryl, halo, OR₈, and cyano; and
R₁₅ and R₁₆ are each independently selected from the group consisting of hydrogen, optionally substituted alkyl, optionally substituted cycloalkyl, optionally substituted aryl, halo, perhaloalkyl, —OR₈, —NO₂, —N(R₈)₂, —NHC(═O)R₈, —NH(SO₂)Ar, —(CR₉R₁₀)ₘ—S(═O)—(CR₉R₁₀)ₚ—R₈, —(CR₉R₁₀)ₙ—S(═O)₂—(CR₉R₁₀)ₚ—R₈, and cyano, wherein Ar is an optionally substituted aryl, and m and p is each independently 0-10, inclusive (i.e., m or p is selected from the group consisting of 0, 1, 2, 3, 4, 5, 6, 7, 8, 9, 10);
R₈ is selected from the group consisting of hydrogen, optionally substituted alkyl, optionally substituted cycloalkyl, and optionally substituted aryl;
R₉ and R₁₀ are each independently selected from the group consisting of hydrogen, optionally substituted alkyl, optionally substituted cycloalkyl, and optionally substituted aryl;
R₁₁ is selected from the group consisting of optionally substituted aryl, and optionally substituted heteroaryl;
R₁₂ is selected from the group consisting of hydrogen, optionally substituted alkyl, optionally substituted cycloalkyl, optionally substituted aryl, and optionally substituted heteroaryl; and
bond a and bond b are a single bond or double bond, such that X₁, X₂, and X₃ have a complete octet along with R₅-R₇.

Also disclosed are pharmaceutical compositions comprising a therapeutically effective amount of at least one compound of Formula I, Formula II, Formula III, Formula IV, or Formula V, or a pharmaceutically acceptable salt, ester, amide, or prodrug thereof, and a physiologically acceptable carrier, diluent, or excipient.

In addition, disclosed are methods of controlling HIF levels in a subject, inhibiting hydroxylation of HIFα in a subject, inhibiting prolyl hydroxylases in a subject, modulating expression of HIF-controlled genes in a subject, treating an HIF-related disorder in a subject, treating diseases associated with ischemia, hypoxia and/or anemia, treating conditions in a subject associated with angiogenesis and/or erythropoietin levels, or treating a disorder in a subject, the method comprising identifying a subject in need thereof and administering to the subject, or contacting the subject with, at least one compound of Formula I, Formula II, Formula III, Formula IV, and Formula V, or a pharmaceutically acceptable salt, ester, amide, or prodrug thereof.

### DETAILED DESCRIPTION OF THE EMBODIMENTS

The term “pharmaceutically acceptable salt” means those salts of compounds of the invention that are safe and effective for use in a subject and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977), incorporated herein by reference.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3.sup.rd Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Whenever a group of this invention is described as being “optionally substituted” that group may be unsubstituted or substituted with one or more of the substituents described for that group. Likewise, when a group is described as being “unsubstituted or substituted,” if substituted, the substituent may be selected from the same group of substituents. Unless otherwise indicated, when a substituent is deemed to be “optionally substituted,” or “substituted” it is meant that the substitutent is a group that may be substituted with one or more group(s) individually and independently selected from alkyl, alkenyl, allynyl, cycloalkyl, cycloalkenyl, cycloalkynyl, aryl, heteroaryl, heteroalicyclyl, aralkyl, heteroaralkyl, (hetereoalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. The protecting groups that may form the protective derivatives of the above substituents are known to those of skill in the art and may be found in references Greene and Wuts, Protective Groups in Organic Synthesis, 3^{rd} Ed., John Wiley & Sons, New York, N.Y., 1999, which is hereby incorporated by reference in its entirety.

As used herein, “Cₘ-Cₙ” in which “m” and “n” are integers refers to the number of carbon atoms in an alkyl, alkenyl or alkynyl group or the number of carbon atoms in the ring of a cycloalkyl, cycloalkenyl, or aryl group. That is, the alkyl, alkenyl, alkynyl, ring of the cycloalkyl, ring of the cycloalkenyl, or of the aryl can contain from “m” to “n”, inclusive, carbon atoms. Thus, for example, a “C₁-C₄ alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)—, and (CH₃)₃CH—. If no “m” and “n” are designated with regard to an alkyl, alkenyl, alkynyl, cycloalkyl or cycloalkenyl group, the broadest range described in these definitions is to be assumed.

As used herein, “alkyl” refers to a straight or branched chain fully saturated (no double or triple bonds) hydrocarbon (all carbon) group. An alkyl group of this invention may comprise from 1-20 carbon atoms, that is, “m”=1 and “n”=20, designated as a “C₁ to C₂₀ alkyl.” It is presently preferred that “m”=1 and “n”:=12 (C₁ to C₁₂ alkyl). It is presently more preferred that “m”=1 and “n”=6 (C₁ to C₆ alkyl). Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl and dodecyl.

An alkyl group of this invention may be substituted or unsubstituted. When substituted, the substituent group(s) may be one or more group(s) independently selected from cycloalkyl, aryl, heteroaryl, heteroalicyclyl, hydroxy, alkoxy, aryloxy, mercapto, alkylthio, arylthio, cyano, halo, oxo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, —NR^{a}R^{b}, protected hydroxyl, protected amino, protected carboxy and protected amido groups.

Examples of substituted alkyl groups include, without limitation, 2-oxo-prop-1-yl, 3-oxo-but-1-yl, cyanomethyl, nitromethyl, chloromethyl, hydroxymethyl, tetrahydropyranyloxymethyl, m-trityloxymethyl, propionyloxymethyl, aminomethyl, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichlorobutyl, 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, N-acetyl-2-aminoethyl, N-benzoyl-1-amino ethyl and N-acetyl-1-aminoethyl.

As used herein, “alkenyl” refers to an alkyl group that contains in the straight or branched hydrocarbon chain one or more double bonds. Examples of alkenyl groups include, without limitation, vinyl (CH₂═CH—), allyl (CH₃CH═CH₂—), 1-propenyl, 2-propenyl, 1-butenyl, 2-butenyl; 1-pentenyl, 2-pentenyl, 3-pentenyl, 4-pentenyl, 3-methyl-1-butenyl, and the various isomers of hexenyl, heptenyl, octenyl, nonenyl, decenyl undecenyl and dodecenyl.

An alkenyl group of this invention may be unsubstituted or substituted. When substituted, the substituent(s) may be selected from the same groups disclosed above with regard to alkyl group substitution. Examples of substituted alkenyl groups include, without limitation, styrenyl, 3-chloro-propen-1-yl, 3-chloro-buten-1-yl, 3-methoxy-propen-2-yl, 3-phenyl-buten-2-yl and 1-cyano-buten-3-yl.

As used herein, “alkynyl” refers to an alkyl group that contains in the straight or branched hydrocarbon chain one or more triple bonds.

An alkynyl group of this invention may be unsubstituted or substituted. When substituted, the substituent(s) may be selected from the same groups disclosed above with regard to alkyl group substitution.

As used herein, “cycloalkyl” refers to a completely saturated (no double bonds) hydrocarbon ring. Cycloalkyl groups of this invention may range from C₃ to C₈. A cycloalkyl group may be unsubstituted or substituted. If substituted, the substituent(s) may be selected from those indicated above with regard to substitution of an alkyl group. The “cycloalkyl” group can be made up of two or more fused rings (rings that share two adjacent carbon atoms). When the cycloalkyl is a fused ring system, then the ring that is connected to the rest of the molecule is a cycloalkyl as defined above. The other ring(s) in the fused ring system may be a cycloallcyl, a cycloalkenyl, an aryl, a heteroaryl, or a heteroalicyclic.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). A cycloalkenyl group of this invention may unsubstituted or substituted. When substituted, the substituent(s) may be selected from the same groups disclosed above with regard to alkyl group substitution. The “cycloalkenyl” group can be made up of two or more fused rings (rings that share two adjacent carbon atoms). When the cycloalkenyl is a fused ring system, then the ring that is connected to the rest of the molecule is a cycloalkenyl as defined above. The other ring(s) in the fused ring system may be a cycloalkyl, a cycloalkenyl, an aryl, a heteroaryl, or a heteroalicyclic.

The term “alkylene” refers to an alkyl group, as defined here, which is a biradical and is connected to two other moieties. Thus, methylene (—CH₂—), ethylene (—CH₂CH₂—), propylene (—CH₂CH₂CH₂—), isopropylene (—CH₂—CH(CH₃)—), and isobutylene (—CH₂—CH(CH₃)—CH₂—) are examples, without limitation, of an alkylene group. Similarly, the term “cycloalkylene” refers to a cycloalkyl group, as defined here, which binds in an analogous way to two other moieties. If the alkyl and cycloalkyl groups contain unsaturated carbons, the terms “alkenylene” and “cycloalkenylene” are used.

As used herein, “acyl” refers to an “RC(═O)O—” Examples of acyl groups include, without limitation, formyl, acetyl, propionyl, butyryl, pentanoyl, pivaloyl, hexanoyl, heptanoyl, octanoyl, nonanoyl, decanoyl, undecanoyl, dodecanoyl and benzoyl. Presently preferred acyl groups are acetyl and benzoyl.

An acyl group of this invention may be unsubstituted or substituted. When substituted, the substituent(s) may be selected from the same groups disclosed above with regard to alkyl group substitution. Example of substituted acyl groups include, without limitation, 4-phenylbutyroyl, 3-phenylbutyroyl, 3-phenylpropanoyl, 2-cyclohexanylacetyl, cyclohexanecarbonyl, 2-furanoyl and 3-dimethylaminobenzoyl.

As used herein, “aryl” refers to a carbocyclic (all carbon) ring that has a fully delocalized pi-electron system. The “aryl” group can be made up of two or more fused rings (rings that share two adjacent carbon atoms). When the aryl is a fused ring system, then the ring that is connected to the rest of the molecule has a fully delocalized pi-electron system. The other ring(s) in the fused ring system may or may not have a fully delocalized pi-electron system. Examples of aryl groups include, but are not limited to, benzene, naphthalene and azulene.

As used herein, “heteroaryl” refers to a ring that contains one or more heteroatoms selected from the group consisting of nitrogen, oxygen and sulfur in the ring and that has a fully delocalized pi-electron system. The “heteroaryl” group can be made up of two or more fused rings (rings that share two adjacent carbon atoms). When the heteroaryl is a fused ring system, then the ring that is connected to the rest of the molecule has a fully delocalized pi-electron system. The other ring(s) in the fused ring system may or may not have a fully delocalized pi-electron system. Examples of heteroaryl rings include, but are not limited to, furan, thiophene, phthalazinone, pyrrole, oxazole, thiazole, imidazole, pyrazole, isoxazole, isothiazole, triazole, thiadiazole, pyran, pyridine, pyridazine, pyrimidine, pyrazine and triazine.

As used herein, “heterocycloalkyl,” “heteroalicyclic,” or “heteroalicyclyl” refers to a ring having in the ring system one or more heteroatoms independently selected from nitrogen, oxygen and sulfur. The ring may also contain one or more double bonds provided that they do not form a fully delocalized pi-electron system in the rings. Heteroalicyclyl groups of this invention may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethanesulfonamido. The “heterocycloalkyl” group can be made up of two or more fused rings (rings that share two adjacent carbon atoms). When the heterocycloalkyl is a fused ring system, then the ring that is connected to the rest of the molecule is a heterocycloalkyl as defined above. The other ring(s) in the fused ring system may be a cycloalkyl, a cycloalkenyl, an aryl, a heteroaryl, or a heteroalicyclic.

As used herein, “phenylalkyl” refers to a phenyl ring covalently bonded to an alkyl group as defined herein. Examples, without limitation, of phenylalkyl groups include, without limitation, benzyl, 2-phenylethyl, 1-phenylpropyl, 4-phenylhexyl, 3-phenylamyl and 3-phenyl-2-methylpropyl. Presently preferred phenylalkyl groups are those wherein the phenyl group is covalently bonded to one of the presently preferred alkyl groups. A phenyl alkyl group of this invention may be unsubstituted or substituted. Examples of substituted phenylalkyl groups include, without limitation, 2-phenyl-1-chloroethyl, 2-(4-methoxyphenyl)ethyl, 4-(2,6-dihydroxy phenyl)hexyl, 2-(5-cyano-3-methoxyphenyl)pentyl, 3-(2,6-dimethylphenyl)propyl, 4-chloro-3-aminobenzyl, 6-(4-methoxyphenyl)-3-carboxy(n-hexyl), 5-(4-aminomethylphenyl)-3-(aminomethyl)pentyl and 5-phenyl-3-oxo-pent-1-yl.

As used herein, “heteroarylalkyl” and “heteroalicyclylalkyl” refer to a heteroaryl or a heteroalicyclyl group covalently bonded to an alkyl group, as defined herein. Examples of such groups include, without limitation, 2-pyridylethyl, 3-pyridylpropyl, 4-furylhexyl, 3-piperazylamyl and 3-morpholinylbutyl. Presently preferred heteroarylalkyl and heteroalicyclylalkyl groups are those in which a presently preferred heteroaryl or heteroalicyclyl group is covalently bonded to a presently preferred alkyl group as disclosed herein.

As used herein, “phenyl” refers to a 6-member aryl group. A phenyl group may be unsubstituted or substituted. When substituted the substituent(s) is/are one or more, preferably one or two, group(s) independently selected from the group consisting of halogen, hydroxy, protected hydroxy, cyano, nitro, alkyl, alkoxy, acyl, acyloxy, carboxy, protected carboxy, carboxymethyl, protected carboxymethyl, hydroxymethyl, protected hydroxymethyl, —NR^{a}R^{b} wherein R^{a} and R^{b} are as defined above but in addition R^{a} may be an amino protecting group as defined herein, carboxamide, protected carboxamide, N-alkylcarboxamide, protected N-alkylcarboxamide, N,N-dialkylcarboxamide, trifluoromethyl, N-alkylsulfonylamino, N-(phenylsulfonyl)amino and phenyl (resulting in the formation of a biphenyl group).

Examples of substituted phenyl groups include, without limitation, 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 and 4-fluorophenyl, 2, 3 or 4-hydroxyphenyl, 2,4-dihydroxyphenyl, the protected-hydroxy derivatives thereof, 2, 3 or 4-nitrophenyl; 2, 3 or 4-cyanophenyl; 2, 3 or 4-methylphenyl, 2,4-dimethylphenyl, 2, 3 or 4-(iso-propyl)phenyl, 2, 3 or 4-ethylphenyl, 2, 3 or 4-(n-propyl)phenyl, 2,6-dimethoxyphenyl, 2, 3 or 4-methoxyphenyl, 2, 3 or 4-ethoxyphenyl, 2, 3 or 4-(isopropoxy)phenyl, 2, 3 or 4-(t-butoxy)phenyl, 3-ethoxy-4-methoxyphenyl; 2, 3 or 4-trifluoromethylphenyl; 2, 3 or 4-carboxyphenyl or 2,4-di(protected carboxy)phenyl; 2, 3, or 4-(protected hydroxymethyl)phenyl or 3,4-di(hydroxymethyl)phenyl; 2, 3 or 4-(aminomethyl)phenyl or 2,4-(protected aminomethyl)phenyl; and 2, 3 or 4-(N-(methylsulfonylamino))phenyl.

As used herein, “phenylalkoxy” refers to a “phenylalkyl-O—” group with “phenyl” and “alkyl” as defined herein. A phenylalkoxy group of this invention may be substituted or unsubstituted on the phenyl ring, in the alkyl group or both. Examples of phenylalkoxy groups include, without limitation, 2-(4-hydroxyphenyl)ethoxy, 4-(4-methoxyphenyl)butoxy, (2R)-3-phenyl-2-amino-propoxy, (2S)-3-phenyl-2-amino-propoxy, 2-indanoxy, 6-phenyl-1-hexanoxy, cinnamyloxy, 2-phenyl-1-propoxy and 2,2-dimethyl-3-phenyl-1-propoxy.

As used herein, “halo” and “halogen” refer to the fluoro, chloro, bromo or iodo atoms. Preferred halogens are chloro and fluoro.

As used herein, “amino protecting group” refers to a group commonly employed to keep (i.e., to “block” or “protect”) an amino group from reacting with a reagent while it reacts with an intended target functional group of a molecule.

As used herein, a “protected carboxamide” refers to a carboxamide in which the nitrogen is substituted with an amino protecting group.

Examples of amino protecting groups include, without limitation, formyl (“For”), trityl, phthalimido, trichloroacetyl, chloroacetyl, bromoacetyl, iodoacetyl groups, t-butoxycarbonyl (“Boc”), 2-(4-biphenylyl)propyl-2-oxycarbonyl (“Bpoc”), 2-phenylpropyl-2-oxycarbonyl (“Poc”), 2-(4-xenyl)isopropoxycarbonyl, 1,1-diphenylethyl-1-oxycarbonyl, 1,1-diphenylpropyl-1-oxycarbonyl, 2-(3,5-dimethoxyphenyl)propyl-2-oxycarbonyl (“Ddz”), 2-(p-toluoyl)propyl-2-oxycarbonyl, cyclopentanyloxycarbonyl, 1-methylcyclopentanyloxycarbonyl, cyclohexanyloxy-carbonyl, 1-methylcyclohexanyloxycarbonyl, 2-methylcyclohexanyloxycarbonyl, 2-(4-toluoylsulfonyl)-ethoxycarbonyl, 2-(methylsulfonyl)ethoxycarbonyl, 2-(triphenylphosphino)-ethoxycarbonyl, 9-fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyl-oxycarbonyl, 2,2,2-trichloroethoxycarbonyl, 2-ethynyl-2-propoxycarbonyl, cyclopropyl-methoxycarbonyl, isobornyloxycarbonyl, 1-piperidyloxycarbonyl, benzyloxycarbonyl (“Cbz”), 4-phenylbenzyloxycarbonyl, 2-methylbenzyloxy-carbonyl, -2,4,5,-tetramethylbenzyloxycarbonyl (“Tmz”), 4-methoxybenzyloxy-carbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyl-oxycarbonyl, 4-bromobenzyloxycarbonyl, 3-bromobenzyloxycarbonyl, 4-nitrobenzyloxy-carbonyl, 4-cyanobenzyloxycarbonyl, 4-(decyloxy)benzyloxycarbonyl, benzoylmethylsulfonyl, dithiasuccinoyl (“Dts”), 2-(nitro)phenylsulfenyl (“Nps”), and diphenyl-phosphine oxide. The species of amino-protecting group employed is not critical so long as the derivatized amino group is stable to the conditions of the subsequent reaction(s) and can be removed at the appropriate point without disrupting the remainder of the molecule. Presently preferred amino-protecting groups are Boc, Cbz and Fmoc. Descriptions of these and other amino-protecting groups may be found in T. W. Greene and P. G. M. Wuts, “Protective Groups in Organic Synthesis,” 2nd ed., John Wiley and Sons, New York, N.Y., 1991, Chapter 7, M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984.

As used herein, the term “carboxy protecting group” refers to a labile ester commonly used to block or protect a carboxylic acid while reactions are carried out on other functional groups on the compound. Examples of carboxy protecting groups include, without limitation, t-butyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxytrityl, 4,4′,4″-trimethoxytrityl, 2-phenylpropyl, trimethylsilyl, t-butyldimethylsilyl, phenacyl, 2,2,2-trichloroethyl, -(trimethylsilyl)ethyl, -(di(n-butyl)methylsilyl)ethyl, p-toluenesulfonylethyl, 4-nitrobenzylsulfonylethyl, allyl, cinnamyl, and 1-(trimethylsilylmethyl)-propenyl. The ester employed is not critical so long as it is stable to the conditions of subsequent reaction(s) and can be removed at the appropriate point without disrupting the remainder of the molecule. Further examples of carboxy-protecting groups are found in E. Haslam, “Protective Groups in Organic Chemistry,” J. G. W. McOmie, Ed., Plenum Press, New York, N.Y., 1973, Chapter 5, and T. W. Greene and P. G. M. Wuts, “Protective Groups in Organic Synthesis,” 2nd ed., John Wiley and Sons, New York, N.Y., 1991, Chapter 5.

As used herein, a “hydroxyl protecting group” refers to a readily cleavable group that replaces the hydrogen of the hydroxyl group, such as, without limitation, tetrahydropyranyl, 2-methoxypropyl, 1-ethoxyethyl, methoxymethyl, 2-methoxyethoxymethyl, methylthiomethyl, t-butyl, t-amyl, trityl, 4-methoxytrityl, 4,4′-dimethoxytrityl, 4,4′,4″-trimethoxytrityl, benzyl, allyl, trimethylsilyl, (t-butyl)dimethylsilyl, and 2,2,2-trichloroethoxycarbonyl. The species of hydroxy-protecting groups is not critical so long as the derivatized hydroxyl group is stable to the conditions of subsequent reaction(s) and can be removed at the appropriate point without disrupting the remainder of the molecule. Further examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2nd ed., John Wiley and Sons, New York, N.Y., 1991, Chapters 2 and 3.

As used herein, “alkylthio” refers to an “alkyl-S—” group, with alkyl as defined above. Examples of alkylthio group include, without limitation, methylthio, ethylthio, n-propylthio, isopropylthio, n-butylthio and t-butylthio.

As used herein, “alkylsulfinyl” refers to an “alkyl-SO—” group, with alkyl as defined above. Examples of alkylsulfinyl groups include, without limitation, methylsulfinyl, ethylsulfinyl, n-propylsulfinyl, isopropylsulfinyl, n-butylsulfinyl and sec-butylsulfinyl.

As used herein, “alkylsulfonyl” refers to an “alkyl-SO₂—” group. Examples of alkylsulfonyl groups include, without limitation, methylsulfonyl, ethylsulfonyl, n-propylsulfonyl, isopropylsulfonyl, n-butylsulfonyl, and t-butylsulfonyl.

As used herein, “phenylthio,” “phenylsulfinyl,” and “phenylsulfonyl” refer to a “phenyl-S—,” “phenyl-SO—,” and “phenyl-SO₂—” group, with phenyl as defined herein.

As used herein, “alkylaminocarbonyl” refers to an “alkylNHC(═O)—” group, with alkyl as defined herein. Examples of alkylaminocarbonyl groups include, without limitation, methylaminocarbonyl, ethylaminocarbonyl, propylaminocarbonyl and butylaminocarbonyl. Examples of substituted alkylaminocarbonyl include, without limitation, methoxymethyl-aminocarbonyl, 2-chloroethylaminocarbonyl, 2-oxopropylaminocarbonyl and 4-phenylbutylaminocarbonyl.

As used herein, “alkoxycarbonyl” refers to an “alkyl-OC(═O)—” group, with alkyl as defined above.

As used herein, “phenylaminocarbonyl” refers to a “phenyl-NHC(═O)—” group, with phenyl as defined above. Examples of substituted phenylaminocarbonyl groups include, without limitation, 2-chlorophenyl-aminocarbonyl, 3-chlorophenylaminocarbonyl, 2-nitorphenylaminocarbonyl, 4-biphenylaminocarbonyl, and 4-methoxyphenylaminocarbonyl.

As used herein, “alkylaminothiocarbonyl” refers to an “alkyl-NHC(═O)—” group, with alkyl as defined above. Examples of alkylaminothio-carbonyl groups include, without limitation, methylaminothiocarbonyl, ethylaminothiocarbonyl, propylaminothiocarbonyl and butylaminothiocarbonyl.

Examples of alkyl-substituted alkylaminothiocarbonyl groups include, without limitation, methoxymethylaminothiocarbonyl, 2-chloroethylaminothiocarbonyl, 2-oxopropylaminothiocarbonyl and 4-phenylbutylaminothiocarbonyl.

As used herein, “phenylaminothiocarbonyl” refers to a “phenyl-NHC(═S)—” group, with phenyl as defined above. Examples of phenylaminothiocarbonyl groups include, without limitation, 2-chlorophenylaminothiocarbonyl, 3-chlorophenyl-aminothiocarbonyl, 2-nitrophenylaminothiocarbonyl, 4-biphenylaminothiocarbonyl and 4-methoxyphenylaminothiocarbonyl.

As used herein, “carbamoyl” refers to an “—NCO—” group.

As used herein, “hydroxyl” refers to an “—OH” group.

As used herein, “cyano” refers to a “—C≡N” group.

As used herein, “nitro” refers to an “—NO₂” group.

An “O-carboxy” group refers to a “RC(═O)O—” group with R as defined above.

A “C-carboxy” group refers to a “—C(═O)OR” group with R as defined above.

An “acetyl” group refers to a CH₃C(═O)— group.

A “trihalomethanesulfonyl” group refers to an “X₃CSO₂—” group wherein X is a halogen.

An “isocyanato” group refers to an “—NCO” group.

A “thiocyanato” group refers to a “—CNS” group.

An “isothiocyanato” group refers to an “—NCS” group.

A “sulfinyl” group refers to an “—S(═O)—R” group with R as defined above.

An “S-sulfonamido” group refers to a “—SO₂NR” group with R as defined above.

An “N-sulfonamido” group refers to a “RSO₂NH—” group with R as defined above.

A “trihalomethanesulfonamido” group refers to an “X₃CSO₂NR—” group with X as halogen and R as defined above.

An “O-carbamyl” group refers to a “—OC(═O)—NR” group with R as defined above.

An “N-carbamyl” group refers to an “ROC(═O)NH—” group with R as defined above.

An “O-thiocarbamyl” group refers to a “—OC(═S)—NR” group with R as defined above.

“N-thiocarbamyl” group refers to an “ROC(═S)NH—” group with R as defined above.

A “C-amido” group refers to a “—C(═O)—NR^{a}R^{b} group with R^{a} and R^{b} as defined above.

An “N-amido” group refers to a RC(═O)NH— group with R as defined above.

The term “perhaloalkyl” refers to an alkyl group in which all the hydrogen atoms are replaced by halogen atoms.

As used herein, an “ester” refers to a “C(O)OR^{a}” group with R^{a} as defined herein.

As used herein, an “amide” refers to a “C(O)NR^{a}R^{b}” group with R^{a} and R^{b} as defined herein.

Any unsubstituted or monosubstituted amine group on a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999). Compounds containing any such converted hydroxyl, amino and/or carboxylic acid groups are within the scope of this invention.

As used herein, an “ether” refers to an “—C—O—C—” group wherein either or both carbons may independently be part of an alkyl, alkenyl, alkynyl, aryl, heteroaryl or heteroalicyclyl group.

As used herein, a “halogenated ether” refers to an ether in which the groups to either side of the oxygen are both alkyl substituted with halogen.

As used herein, “amino acid” refers to any one of the twenty naturally-occurring L-amino acids, to their non-natural D-enantiomers, to non-naturally occurring amino acids such as, without limitation, norleucine (“Nle”), norvaline (“Nva”), L- or D-naphthalanine, ornithine (“Orn”), homoarginine (homoArg) and to other amino acids well-known in the peptide art such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill.

Amino acids are referred to herein by their full chemical names, by their three letter codes, or by their one letter code, which are well-known to those skilled in the art. Unless the chirality of an amino acid is specifically designated or the amino acid is expressly stated to be a naturally occurring (i.e., L-) amino acid, the amino acid may be D or L or a racemic mixture of the two.

As used herein, a “functionalized resin” refers to any resin to which functional groups have been appended. Such functionalized resins are well-known to those skilled in the art and include, without limitation, resins functionalized with amino, alkylhalo, formyl or hydroxy groups. Examples of functionalized resins which can serve as solid supports for immobilized solid phase synthesis are well-known in the art and include, without limitation, 4-methylbenzhydrylamine-copoly(styrene-1% divinylbenzene) (MBHA), 4-hydroxymethylphenoxymethyl-copoly(styrene-1% divinylbenzene), 4-oxymethyl-phenyl-acetamido-copoly(stryene-1% divinylbenzene) (Wang), 4-(oxymethyl)-phenylacetamido methyl (Pam), and Tentagel™, from Rapp Polymere Gmbh, trialkoxy-diphenyl-methyl ester-copoly(styrene-1% divinylbenzene) (RINK) all of which are commercially available. Other functionalized resins useful in the synthesis of the compounds of this invention will become apparent to those skilled in the art based on the disclosures herein. All such resins are within the scope of this invention.

When two substituents taken together along with the carbon atoms to which they are attached form a five- or six-membered optionally substituted carbocyclic ring or optionally substituted heterocyclic ring, or form a six-membered optionally substituted aryl, optionally substituted heteroaryl, it is meant that the following structure:

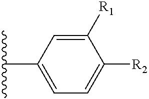

can be representative of, for example, the following structures:

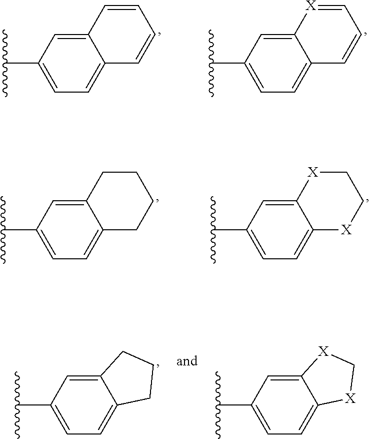

where X is a heteroatom.

Throughout the present disclosure, when a particular compound comprises a chiral center, the scope of the present disclosure also includes compositions comprising the racemic mixture of the two enantiomers, as well as compositions comprising each enantiomer individually substantially free of the other enantiomer. Thus, for example, contemplated herein is a composition comprising the S enantiomer substantially free of the R enantiomer, or a composition comprising the R enantiomer substantially free of the S enantiomer. By “substantially free” it is meant that the composition comprises less than 10%, or less than 8%, or less than 5%, or less than 3%, or less than 1% of the minor enantiomer. If the particular compound comprises more than one chiral center, the scope of the present disclosure also includes compositions comprising a mixture of the various diastereomers, as well as compositions comprising each diastereomer substantially free of the other diastereomers. The recitation of a compound, without reference to any of its particular diastereomers, includes compositions comprising all four diastereomers, compositions comprising the racemic mixture of R,R and S,S isomers, compositions comprising the racemic mixture of R,S and S,R isomers, compositions comprising the R,R enantiomer substantially free of the other diastereomers, compositions comprising the S,S enantiomer substantially free of the other diastereomers, compositions comprising the R,S enantiomer substantially free of the other diastereomers, and compositions comprising the S,R enantiomer substantially free of the other diastereomers.

When a tautomer of the compound of the Formula I exists, the present invention includes any possible tautomers and pharmaceutically acceptable salts thereof, and mixtures thereof, except where specifically drawn or stated otherwise The disclosure and claims of the present invention are based on the known general principles of chemical bonding. It is understood that the claims do not encompass structures known to be unstable or not able to exist based on the literature.

### Compounds

In one aspect, disclosed herein are compounds of Formula I:

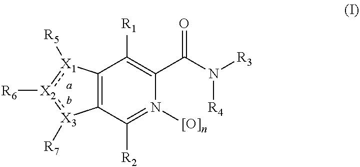

or a pharmaceutically acceptable salt, ester, amide, or prodrug thereof,
wherein
n is 0 or 1;
R₁ is —OR₈ or halo;
R₂ is selected from the group consisting of hydrogen, optionally substituted alkyl, optionally substituted cycloalkyl, optionally substituted aryl, halo, and cyano;
R₃ is selected from the group consisting of optionally substituted cycloalkyl, optionally substituted aryl, optionally substituted heteroaryl, —CR₉R₁₀R₁₁, and —CR₉R₁₀—C(═O)OR₁₂;
R₄ is hydrogen or —OR₈;
R₈ is selected from the group consisting of hydrogen, optionally substituted alkyl, optionally substituted cycloalkyl, and optionally substituted aryl;
R₉ and R₁₀ are each independently selected from the group consisting of hydrogen, optionally substituted alkyl, optionally substituted cycloalkyl, and optionally substituted aryl;
R₁₁ is selected from the group consisting of optionally substituted aryl, and optionally substituted heteroaryl;
R₁₂ is selected from the group consisting of hydrogen, optionally substituted alkyl, optionally substituted cycloalkyl, optionally substituted aryl, and optionally substituted heteroaryl; and
i) X₁ is sulfur;
   R₅ does not exist;
   X₂ and X₃ are both carbon;
   R₆ and R₇ taken together along with the carbon atoms to which they are attached form a ring of formula

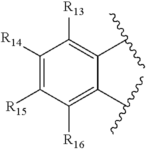

R₁₃ and R₁₄ are each independently selected from the group consisting of hydrogen, optionally substituted alkyl, optionally substituted cycloalkyl, optionally substituted aryl, halo, —OR₈, and cyano;
R₁₅ and R₁₆ are each independently selected from the group consisting of hydrogen, optionally substituted alkyl, optionally substituted cycloalkyl, optionally substituted aryl, halo, perhaloalkyl, —OR₈, —NO₂, —N(R₈)₂, —NHC(═O)R₈, —NH(SO₂)Ar, —(CR₉R₁₀)ₘ—S(═O)—(CR₉R₁₀)ₚ—R₈, —(CR₉R₁₀)ₘ—S(═O)₂—(CR₉R₁₀)ₚ—R₈, and cyano, wherein Ar is an optionally substituted aryl, and m and p is each independently 0-10, inclusive; and
bond a is a single bond and bond b is a double bond; or
ii) X₁ is oxygen;
   R₅ does not exist;
   X₂ and X₃ are both carbon;
   R₅ is selected from the group consisting of hydrogen, optionally substituted alkyl, optionally substituted cycloalkyl, optionally substituted aryl, optionally substituted heteroaryl, and —OR₈;
   R₇ is selected from the group consisting of hydrogen, optionally substituted alkyl, optionally substituted cycloalkyl, optionally substituted aryl, optionally substituted heteroaryl, and —SO₂—Ar, wherein Ar is an optionally substituted aryl; or
   R₆ and R₇ taken together along with the carbon atoms to which they are attached form an optionally substituted phenyl; and
   bond a is a single bond and bond b is a double bond; or
iii) X₁ is carbon and X₂ and X₃ are both nitrogen;
   R₅ is selected from the group consisting of hydrogen, optionally substituted alkyl, optionally substituted cycloalkyl, optionally substituted aryl, and optionally substituted heteroaryl;
   R₆ does not exist;
   R₇ is selected from the group consisting of hydrogen, optionally substituted alkyl, optionally substituted cycloalkyl, optionally substituted aryl, optionally substituted heteroaryl, and —SO₂—Ar, wherein Ar is an optionally substituted aryl; and
   bond a is a single bond and bond b is a double bond.

In another aspect, disclosed herein are compounds of Formula I:

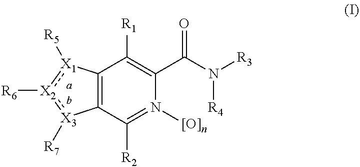

or a pharmaceutically acceptable salt, ester, amide, or prodrug thereof,
wherein
n is 0 or 1;
R₁ is —OR₈ or halo;
R₂ is selected from the group consisting of hydrogen, optionally substituted alkyl, optionally substituted cycloalkyl, optionally substituted aryl, halo, and cyano;
R₃ is selected from the group consisting of optionally substituted cycloalkyl, optionally substituted aryl, optionally substituted heteroaryl, —CR₉R₁₀R₁₁, and —CR₉R₁₀—(═O)OR₁₂;
R₄ is hydrogen or —OR₈;
X₁ is selected from the group consisting of oxygen, sulfur, and carbon;
R₅ is selected from the group consisting of hydrogen, optionally substituted alkyl, optionally substituted cycloalkyl, optionally substituted aryl, and optionally substituted heteroaryl,
   provided that R₅ does not exist when X₁ is oxygen or sulfur;
X₂ and X₃ are each independently nitrogen or carbon,
   provided that at least one of X₁, X₂, and X₃ is carbon;
R₆ is selected from the group consisting of hydrogen, optionally substituted alkyl, optionally substituted cycloalkyl, optionally substituted aryl, optionally substituted heteroaryl, and —OR₈;
R₇ is selected from the group consisting of hydrogen, optionally substituted alkyl, optionally substituted cycloalkyl, optionally substituted aryl, optionally substituted heteroaryl, and —SO₂—Ar, wherein Ar is an optionally substituted aryl; or
X₂ and X₃ are both carbon and R₆ and R₇ taken together along with the carbon atoms to which they are attached form a ring of formula

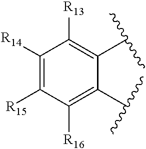

wherein
R₁₃ and R₁₄ are each independently selected from the group consisting of hydrogen, optionally substituted alkyl, optionally substituted cycloalkyl, optionally substituted aryl, halo, —OR₈, and cyano; and
R₁₅ and R₁₆ are each independently selected from the group consisting of hydrogen, optionally substituted alkyl, optionally substituted cycloalkyl, optionally substituted aryl, halo, perhaloalkyl, —OR₈, —NO₂, —N(R₈)₂, —NHC(═O)R₈, —NH(SO₂)Ar, —(CR₉R₁₀)ₚ—S(═O)—(CR₉R₁₀)ₚ—R₈, —(CR₉R₁₀)ₘ—S(═O)₂—(CR₉R₁₀)ₚ—R₈, and cyano, wherein Ar is an optionally substituted aryl, and m and p is each independently 0-10, inclusive;
R₈ is selected from the group consisting of hydrogen, optionally substituted alkyl, optionally substituted cycloalkyl, and optionally substituted aryl;
R₉ and R₁₀ are each independently selected from the group consisting of hydrogen, optionally substituted alkyl, optionally substituted cycloalkyl, and optionally substituted aryl;
R₁₁ is selected from the group consisting of optionally substituted aryl, and optionally substituted heteroaryl;
R₁₂ is selected from the group consisting of hydrogen, optionally substituted alkyl, optionally substituted cycloalkyl, optionally substituted aryl, and optionally substituted heteroaryl; and
bond a and bond b are single bond or double bond, such that X₁, X₂, and X₃ have a complete octet along with R₅-R₇.

In some embodiments, R₁ is selected from the group consisting of fluoro, chloro, bromo, and iodo. In some of these embodiments, R₁ is chloro.

In certain embodiments, R₁ is —OR₈ and R₈ is selected from the group consisting of hydrogen, and optionally substituted alkyl. In some of these embodiments, R₈ is selected from the group consisting of hydrogen, methyl, ethyl, n-propyl, isopropyl, n-butyl, sec-butyl, and tert-butyl.

In some embodiments, R₂ is selected from the group consisting of hydrogen, optionally substituted alkyl, fluoro, chloro, bromo, iodo, and cyano. The alkyl group in some of these embodiments is selected from the group consisting of methyl, ethyl, n-propyl, isopropyl, n-butyl, sec-butyl, and tert-butyl. In some embodiments, R₂ is selected from the group consisting of hydrogen, methyl, chloro, bromo, and cyano.

In some embodiments, R₃ is optionally substituted aryl, which can be an optionally substituted phenyl. In some embodiments, R₃ is phenyl. In other embodiments, R₃ is optionally substituted heteroaryl, which can be an optionally substituted pyridyl. In some embodiments, R₃ is pyridyl.

In other embodiments, R₃ is —CR₉R₁₀R₁₁. In some of these embodiments, R₉ is selected from the group consisting of hydrogen, optionally substituted alkyl, and optionally substituted aryl. In some embodiments, R₉ is hydrogen. In some embodiments, R₁₀ is selected from the group consisting of hydrogen, optionally substituted alkyl, and optionally substituted aryl. In some embodiments, R₁₀ is hydrogen or methyl. The alkyl group in the above embodiments can be selected from the group consisting of methyl, ethyl, n-propyl, isopropyl, n-butyl, sec-butyl, and tert-butyl.

In some embodiments, R₁₁ is optionally substituted aryl, which can be an optionally substituted phenyl. In some embodiments, R₁₁ is phenyl. In other embodiments, R₁₁ is optionally substituted heteroaryl, which can be an optionally substituted pyridyl or an optionally substituted tetrazolyl. In some embodiments, R₁₁ is pyridyl, while in other embodiments, R₁₁ is 1H-tetrazol-5-yl. In certain of these embodiments, R₁₁ is [2-(4-methoxy-benzyl)-2H-tetrazol-5-yl.

In other embodiments, R₃ is —CR₉R₁₀—C(═O)OR₁₂. In some embodiments, R₉ and R₁₀ are as described above. In some embodiments, R₁₂ is hydrogen or optionally substituted alkyl, where the alkyl can be selected from the group consisting of methyl, ethyl, n-propyl, isopropyl, n-butyl, sec-butyl, and tert-butyl. In some embodiments, Rᵤ is hydrogen or methyl.

In some embodiments, R₄ is —OR₈ and R₈ can be selected from the group consisting of hydrogen, and optionally substituted alkyl. In certain of these embodiments, R₈ is selected from the group consisting of hydrogen, methyl, ethyl, n-propyl, isopropyl, n-butyl, sec-butyl, and tert-butyl. In some embodiments, R₄ is hydrogen or hydroxyl.

In some embodiments, X₁ is oxygen and R₅ does not exist, while in other embodiments, X₁ is carbon and R₅ is selected from the group consisting of hydrogen, optionally substituted alkyl, optionally substituted cycloalkyl, and optionally substituted aryl. The alkyl in these embodiments can be selected from the group consisting of methyl, ethyl, n-propyl, isopropyl, n-butyl, sec-butyl, and tert-butyl.

In other embodiments, X₁ is carbon and R₅ is hydrogen.

In some embodiments, X₂ is nitrogen and R₆ is selected from the group consisting of hydrogen, optionally substituted alkyl, optionally substituted cycloalkyl, and optionally substituted aryl, where the alkyl can be selected from the group consisting of methyl, ethyl, n-propyl, isopropyl, n-butyl, sec-butyl, and tert-butyl. In some of these embodiments, X₂ is nitrogen and R₆ is hydrogen.

In other embodiments, X₂ is carbon and R₆ is selected from the group consisting of hydrogen, optionally substituted alkyl, optionally substituted cycloalkyl, and optionally substituted aryl, where the alkyl can be selected from the group consisting of methyl, ethyl, n-propyl, isopropyl, n-butyl, sec-butyl, and tert-butyl. In some of these embodiments, X₂ is carbon and R₆ is hydrogen or phenyl.

In some embodiments, X₃ is nitrogen and R₇ is selected from the group consisting of hydrogen, optionally substituted alkyl, optionally substituted aryl, optionally substituted heteroaryl, and —SO₂—Ar, where the alkyl can be selected from the group consisting of methyl, ethyl, n-propyl, isopropyl, n-butyl, sec-butyl, and tert-butyl. In some of these embodiments, the aryl is phenyl. In other embodiments, the heteroaryl is pyridyl. In further embodiments, Ar is phenyl. In some embodiments, R₇ is selected from the group consisting of hydrogen, phenyl, pyridyl, and —SO₂—C₆H₅. In other embodiments, X₃ is carbon and R₇ is as described above. In some of these embodiments, X₃ is carbon and R₇ is hydrogen or phenyl.

Some embodiments are directed to a compound of Formula I, as described above, where

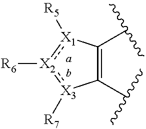

is selected from the group consisting of

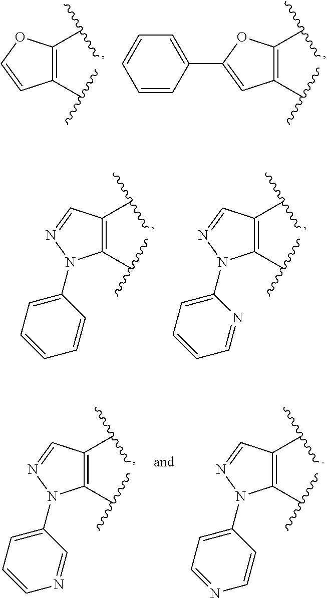

In some embodiments of the compounds of Formula I, X₁ is oxygen; R₅ does not exist; X₂ and X₃ are both carbon; and R₆ and R₇ taken together along with the carbon atoms to which they are attached form an optionally substituted phenyl.

In other embodiments of the compounds of Formula I, X₁ is sulfur; R₅ does not exist; X₂ and X₃ are both carbon; and R₆ and R₇ taken together along with the carbon atoms to which they are attached form a ring of formula

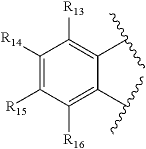

where R₁₃-R₁₆ are as described herein.

In some embodiments, R₁₃ is selected from the group consisting of hydrogen, halogen, optionally substituted alkyl, and optionally substituted aryl. In some of these embodiments, R₁₃ is hydrogen. In other embodiments, R₁₃ is selected from the group consisting of fluoro, chloro, bromo, and iodo. In some embodiments, R₁₄ is selected from the group consisting of hydrogen, halogen, optionally substituted alkyl, and optionally substituted aryl. In some of these embodiments, R₁₄ is hydrogen. In other embodiments, R₁₄ is selected from the group consisting of fluoro, chloro, bromo, and iodo.

In some embodiments, R₁₆ is selected from the group consisting of hydrogen, optionally substituted alkyl, optionally substituted aryl, and —(CR₉R₁₀)ₘ—S(═O)₂—(CR₉R₁₀)ₚ—R₈. In some of these embodiments, m is 0. In other embodiments, m is 1. In some embodiments, p is 0, while in other embodiments p is 1. In some of these embodiments, each R₉ is independently selected from the group consisting of hydrogen, optionally substituted alkyl, and optionally substituted aryl. In some embodiments, each R₉ is independently hydrogen. In some embodiments, each R₁₀ is independently selected from the group consisting of hydrogen, optionally substituted alkyl, and optionally substituted aryl. In some embodiments, each R₁₀ is independently hydrogen. In other embodiments, R₈ is an optionally substituted alkyl, where the alkyl can be selected from the group consisting of methyl, ethyl, n-propyl, isopropyl, n-butyl, sec-butyl, and tert-butyl. In some embodiments, R₁₆ is hydrogen. In other embodiments, R₁₆ is —S(═O)₂—CH₂CH₃.

In some embodiments, R₁₅ is selected from the group consisting of hydrogen, halo, perhaloalkyl, —OR₈, —NO₂, —N(R₈)₂, —NHC(═O)R₈, and —NH(SO₂)Ar.

In some of these embodiments, the halo is selected from the group consisting of fluoro, chloro, bromo, and iodo. In certain embodiments, R₁₅ is fluororo. In some embodiments, the perhalohalkyl is selected from the group consisting of perfluoroalkyl, perchloroalkyl, perbromoalkyl, and periodoalkyl. By “perhaloalkyl” it is meant an alkyl moiety where all of the hydrogen atoms normally present on the alkyl are replaced by a halogen. Thus, for example, a perchloroalkyl is an alkyl moiety where all of the carbon atoms not connected to the rest of the molecule are connected to chorine atoms. In some of these embodiments, the alkyl moiety of the perhaloalkyl substituent is selected from the group consisting of methyl, ethyl, n-propyl, isopropyl, n-butyl, sec-butyl, and tert-butyl. In certain embodiments, R₁₅ is trifluoromethyl.

In other embodiments, R₁₅ is —OR₈. In some of these embodiments, R₈ is selected from the group consisting of hydrogen, methyl, ethyl, n-propyl, isopropyl, n-butyl, sec-butyl, and tert-butyl. In certain embodiments, R₁₅ is —OH.

In some embodiments, R₁₅ is —N(R₈)₂, where each R₈ is independently selected from the group consisting of hydrogen, optionally substituted methyl, optionally substituted ethyl, optionally substituted n-propyl, optionally substituted isopropyl, optionally substituted n-butyl, optionally substituted sec-butyl, and optionally substituted tert-butyl. In some of these embodiments, R₁₅ is selected from the group consisting of —NH₂, —NH(CH₃), —NH(CH₂CH₃), —NH(CH₂—C₆H₅), —N(CH₃)₂, —(CH₂CH₃)₂, and —NⁱPr₂ (—N(CH(CH₃)₂)₂).

In some embodiments, R₁₅ is —NH(SO₂)Ar, where Ar is an optionally substituted phenyl. In some of these embodiments, R₁₅ is —NH(SO₂)—C₆H₅.

In some embodiments, R₁₅ is —NHC(═O)R₈. In some of these embodiments, R₈ is an optionally substituted alkyl, where the alkyl can be selected from the group consisting of methyl, ethyl, n-propyl, isopropyl, n-butyl, sec-butyl, and tert-butyl. In some embodiments, R₁₅ is —NHC(═O)CH₃.

In some embodiments, R₁₅ is —(CR₉R₁₀)ₘ—S(═O)—(CR₉R₁₀)ₚ—R₈. In some of these embodiments, m is 0. In other embodiments, m is 1. In some embodiments, p is 0, while in other embodiments p is 1. In some of these embodiments, each R₉ is independently selected from the group consisting of hydrogen, optionally substituted alkyl, and optionally substituted aryl. In some embodiments, each R₉ is independently hydrogen. In some embodiments, each R₁₀ is independently selected from the group consisting of hydrogen, optionally substituted alkyl, and optionally substituted aryl. In some embodiments, each R₁₀ is independently hydrogen. In some embodiments, R₈ is an optionally substituted aryl. In some of these embodiments, the aryl is phenyl. In some embodiments, R₁₅ is —S(═O)-Ph.

In some embodiments, R₁₅ is —(CR₉R₁₀)ₘ—S(═O)₂—(CR₉R₁₀)ₚ—R₈. In some of these embodiments, m is 0. In other embodiments, m is 1. In some embodiments, p is 0, while in other embodiments p is 1. In some of these embodiments, each R₉ is independently selected from the group consisting of hydrogen, optionally substituted alkyl, and optionally substituted aryl. In some embodiments, each R₉ is independently hydrogen. In some embodiments, each R₁₀ is independently selected from the group consisting of hydrogen, optionally substituted alkyl, and optionally substituted aryl. In some embodiments, each R₁₀ is independently hydrogen. In some embodiments, R₈ is an optionally substituted aryl. In some of these embodiments, the aryl is phenyl. In some embodiments, R₈ is an optionally substituted alkyl, where the alkyl can be selected from the group consisting of methyl, ethyl, n-propyl, isopropyl, n-butyl, sec-butyl, and tert-butyl. In some embodiments, R₁₅ is selected from the group consisting of —S(═O)₂—CH₂CH₃, —S(═O)₂-Ph, and —S(═O)₂—CH₂Ph.

In some embodiments of the compounds of Formula I, R₁₅ is selected from the group consisting of hydrogen, fluororo, trifluoromethyl, —OH, —NH₂, —NH(CH₂CH₃), —NH(CH₂—C₆H₅), —N(CH₃)₂, —N(CH₂CH₃)₂, —NH(SO₂)—C₆H₅, and —NHC(═O)CH₃, —(═O)-Ph, S(═O)₂—CH₂CH₃, S(═O)₂-Ph, and —S(═O)₂—CH₂Ph.

In some embodiments of the compounds of Formula I, the ring of formula

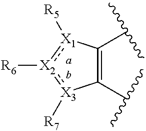

is selected from the group consisting of

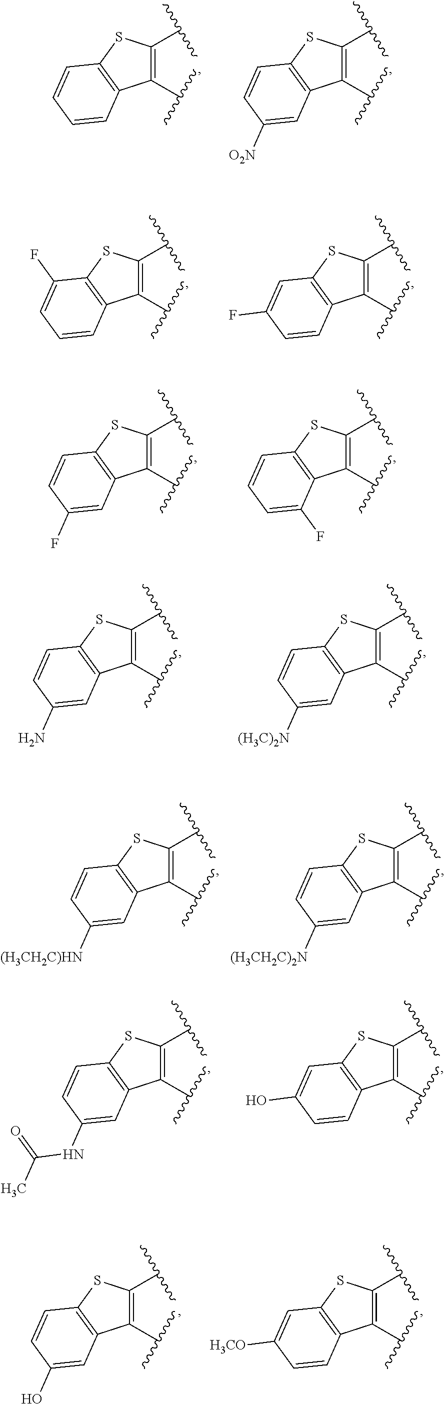

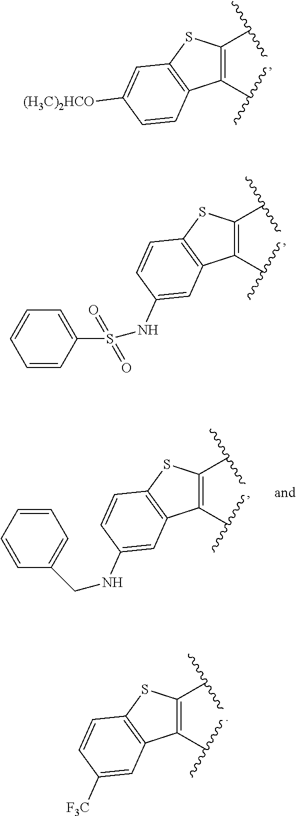

In another aspect, disclosed herein are compounds of Formula II

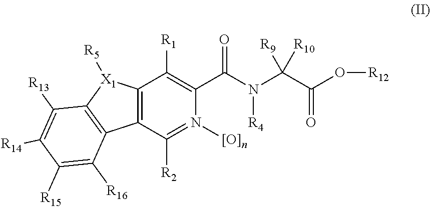

or a pharmaceutically acceptable salt, ester, amide, or prodrug thereof,
where
n is 0 or 1;
R₁ is —OR₈ or halo;
R₂ is selected from the group consisting of hydrogen, optionally substituted alkyl, optionally substituted cycloalkyl, optionally substituted aryl, halo, and cyano;
R₄ is hydrogen or —OR₈;
X₁ is selected from the group consisting of oxygen, sulfur, and carbon;
R₅ is selected from the group consisting of hydrogen, optionally substituted alkyl, optionally substituted cycloalkyl, optionally substituted aryl, and optionally substituted heteroaryl,
   provided that R₅ does not exist when X₁ is oxygen or sulfur;
R₉ and R₁₀ are each independently selected from the group consisting of hydrogen, optionally substituted alkyl, optionally substituted cycloalkyl, and optionally substituted aryl;
R₁₂ is selected from the group consisting of hydrogen, optionally substituted alkyl, optionally substituted cycloalkyl, optionally substituted aryl, and optionally substituted heteroaryl;
R₁₃ and R₁₄ are each independently selected from the group consisting of hydrogen, optionally substituted alkyl, optionally substituted cycloalkyl, optionally substituted aryl, halo, —OR₈, and cyano; and
R₁₅ and R₁₆ are each independently selected from the group consisting of hydrogen, optionally substituted alkyl, optionally substituted cycloalkyl, optionally substituted aryl, halo, perhaloalkyl, —OR₈, —NO₂, —N(R₈)₂, —NHC(═O)R₈, —NH(SO₂)Ar, —(CR₉R₁₀)ₘ—S(═O)—(CR₉R₁₀)ₚ—R₈, (CR₉R₁₀)ₘ—S(═O)₂—(CR₉R₁₀)ₚ—R₈, and cyano, wherein Ar is an optionally substituted aryl, and m and p is each independently 0-10, inclusive.

In another aspect, disclosed herein are compounds of Formula III

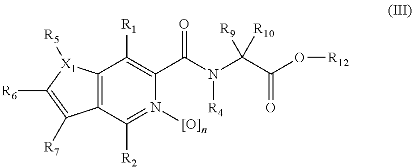

or a pharmaceutically acceptable salt, ester, amide, or prodrug thereof,
where
n is 0 or 1;
R₁ is —OR₈ or halo;
R₂ is selected from the group consisting of hydrogen, optionally substituted alkyl, optionally substituted cycloalkyl, optionally substituted aryl, halo, and cyano;
R₄ is hydrogen or —OR₈;
X₁ is selected from the group consisting of oxygen, sulfur, and carbon;
R₅ is selected from the group consisting of hydrogen, optionally substituted alkyl, optionally substituted cycloalkyl, optionally substituted aryl, and optionally substituted heteroaryl,
   provided that R₅ does not exist when X₁ is oxygen or sulfur;
R₆ is selected from the group consisting of hydrogen, optionally substituted alkyl, optionally substituted cycloalkyl, optionally substituted aryl, optionally substituted heteroaryl, and —OR₈;
R₇ is selected from the group consisting of hydrogen, optionally substituted alkyl, optionally substituted cycloalkyl, optionally substituted aryl, optionally substituted heteroaryl, and —SO₂—Ar, wherein Ar is an optionally substituted aryl;
R₉ and R₁₀ are each independently selected from the group consisting of hydrogen, optionally substituted alkyl, optionally substituted cycloalkyl, and optionally substituted aryl; and
R₁₂ is selected from the group consisting of hydrogen, optionally substituted alkyl, optionally substituted cycloalkyl, optionally substituted aryl, and optionally substituted heteroaryl.

In another aspect, disclosed herein are compounds of Formula IV

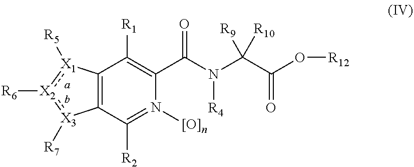

or a pharmaceutically acceptable salt, ester, amide, or prodrug thereof,
where
n is 0 or 1;
R₁ is —OR₈ or halo;
R₂ is selected from the group consisting of hydrogen, optionally substituted alkyl, optionally substituted cycloalkyl, optionally substituted aryl, halo, and cyano;
R₄ is hydrogen or —OR₈;
X₁ is selected from the group consisting of oxygen, sulfur, and carbon;
R₅ is selected from the group consisting of hydrogen, optionally substituted alkyl, optionally substituted cycloalkyl, optionally substituted aryl, and optionally substituted heteroaryl,
   provided that R₅ does not exist when X₁ is oxygen or sulfur;
X₂ and X₃ are each independently nitrogen or carbon,
   provided that at least one of X₁, X₂, and X₃ is carbon;
R₆ is selected from the group consisting of hydrogen, optionally substituted alkyl, optionally substituted cycloalkyl, optionally substituted aryl, optionally substituted heteroaryl, and —OR₈;
R₇ is selected from the group consisting of hydrogen, optionally substituted alkyl, optionally substituted cycloalkyl, optionally substituted aryl, optionally substituted heteroaryl, and —SO₂—Ar, wherein Ar is an optionally substituted aryl;
R₉ and R₁₀ are each independently selected from the group consisting of hydrogen, optionally substituted alkyl, optionally substituted cycloalkyl, and optionally substituted aryl;
R₁₂ is selected from the group consisting of hydrogen, optionally substituted alkyl, optionally substituted cycloalkyl, optionally substituted aryl, and optionally substituted heteroaryl; and
bond a and bond b are single bond or double bond, such that X₁, X₂, and X₃ have a complete octet along with R₅-R₇.

In another aspect, disclosed herein are compounds of Formula V

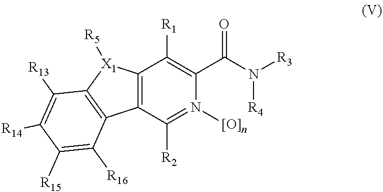

or a pharmaceutically acceptable salt, ester, amide, or prodrug thereof,
where
n is 0 or 1;
R₁ is —OR₈ or halo;
R₂ is selected from the group consisting of hydrogen, optionally substituted alkyl, optionally substituted cycloalkyl, optionally substituted aryl, halo, and cyano;
R₃ is selected from the group consisting of optionally substituted cycloalkyl, optionally substituted aryl, optionally substituted heteroaryl, and —CR₉R₁₀R₁₁;
R₄ is hydrogen or —OR₈;
X₁ is selected from the group consisting of oxygen, sulfur, and carbon;
R₅ is selected from the group consisting of hydrogen, optionally substituted alkyl, optionally substituted cycloalkyl, optionally substituted aryl, and optionally substituted heteroaryl,
   provided that R₅ does not exist when X₁ is oxygen or sulfur;
R₁₃ and R₁₄ are each independently selected from the group consisting of hydrogen, optionally substituted alkyl, optionally substituted cycloalkyl, optionally substituted aryl, halo, —OR₈, and cyano; and
R₁₅ and R₁₆ are each independently selected from the group consisting of hydrogen, optionally substituted alkyl, optionally substituted cycloalkyl, optionally substituted aryl, halo, perhaloalkyl, —OR₈, —NO₂, —N(R₈)₂, —NHC(═O)R₈, —NH(SO₂)Ar, —(CR₉R₁₀)ₘ—S(═O)—(CR₉R₁₀)ₚ—R₈, —(CR₉R₁₀)ₘ—S(═O)₂—(CR₉R₁₀)ₚ—R₈, and cyano, wherein Ar is an optionally substituted aryl, and m and p is each independently 0-10, inclusive.

In another aspect, disclosed herein is a compound selected from the group consisting of
[(4-Hydroxy-benzo[4,5]furo[3,2-c]pyridine-3-carbonyl)-amino]-acetic acid,
[(4-Hydroxy-benzo[4,5]thieno[3,2-c]pyridine-3-carbonyl)-amino]-acetic acid,
[(1-Chloro-4-hydroxy-benzo[4,5]thieno[3,2-c]pyridine-3-carbonyl)-amino]-acetic acid,
[(7-Hydroxy-furo[3,2-c]pyridine-6-carbonyl)-amino]-acetic acid,
[(7-Hydroxy-2-phenyl-furo[3,2-c]pyridine-6-carbonyl)-amino]-acetic acid,
(S)-2-[(7-Hydroxy-furo[3,2-c]pyridine-6-carbonyl)-amino]-propionic acid,
[(4-Hydroxy-1-phenyl-1H-pyrazolo[3,4-c]pyridine-5-carbonyl)-amino]-acetic acid,
[(7-Chloro-4-hydroxy-1-phenyl-1H-pyrazolo[3,4-c]pyridine-5-carbonyl)-amino]-acetic acid,
[(1-Chloro-4-hydroxy-8-nitro-benzo[4,5]thieno[3,2-c]pyridine-3-carbonyl)-amino]-acetic acid,
3-(Carboxymethyl-carbamoyl)-1-chloro-4-hydroxy-benzo[4,5]thieno[3,2-c]pyridin-8-yl-ammonium,
[(1-Bromo-4-hydroxy-benzo[4,5]thieno[3,2-c]pyridine-3-carbonyl)-amino]-acetic acid,
(S)-2-[(1-Chloro-4-hydroxy-benzo[4,5]thieno[3,2-c]pyridine-3-carbonyl)-amino]-propionic acid,
(S)-2-[(1-Bromo-4-hydroxy-benzo[4,5]thieno[3,2-c]pyridine-3-carbonyl)-amino]-propionic acid,
[(1-Chloro-8-fluoro-4-hydroxy-benzo[4,5]thieno[3,2-c]pyridine-3-carbonyl)-amino]-acetic acid,
[(1-Cyano-4-hydroxy-benzo[4,5]thieno[3,2-c]pyridine-3-carbonyl)-amino]-acetic acid,
[(1-Benzenesulfonyl-7-chloro-4-hydroxy-1H-pyrrolo[2,3-c]pyridine-5-carbonyl)-amino]-acetic acid,
[(1-Benzenesulfonyl-7-chloro-4-hydroxy-1H-pyrrolo[2,3-c]pyridine-5-carbonyl)-amino]-acetic acid methyl ester,
[(7-Chloro-4-hydroxy-1H-pyrrolo[2,3-c]pyridine-5-carbonyl)-amino]-acetic acid,
[(4-Amino-1-bromo-benzo[4,5]thieno[3,2-c]pyridine-3-carbonyl)-amino]-acetic acid,
1-Bromo-4-hydroxy-benzo[4,5]thieno[3,2-c]pyridine-3-carboxylic acid (pyridin-3-ylmethyl)-amide,
[(1-Bromo-4-fluoro-benzo[4,5]thieno[3,2-c]pyridine-3-carbonyl)-amino]-acetic acid,
1-Bromo-4-hydroxy-benzo[4,5]thieno[3,2-c]pyridine-3-carboxylic acid [2-(4-methoxy-benzyl)-2H-tetrazol-5-ylmethyl]-amide,
1-Bromo-4-hydroxy-benzo[4,5]thieno[3,2-c]pyridine-3-carboxylic acid [1-(4-methoxy-benzyl)-1H-tetrazol-5-ylmethyl]-amide,
1-Bromo-4-hydroxy-benzo[4,5]thieno[3,2-c]pyridine-3-carboxylic acid (pyridin-2-ylmethyl)-amide,
1-Bromo-4-hydroxy-benzo[4,5]thieno[3,2-c]pyridine-3-carboxylic acid (1H-tetrazol-5-ylmethyl)-amide,
1-Bromo-4-hydroxy-benzo[4,5]thieno[3,2-c]pyridine-3-carboxylic acid pyridin-2-ylamide,
1-Bromo-4-hydroxy-benzo[4,5]thieno[3,2-c]pyridine-3-carboxylic acid pyridin-3-ylamide,
1-Bromo-4-hydroxy-benzo[4,5]thieno[3,2-c]pyridine-3-carboxylic acid phenylamide,
1-Bromo-4-hydroxy-benzo[4,5]thieno[3,2-c]pyridine-3-carboxylic acid benzylamide,
[(1-Chloro-8-dimethylamino-4-hydroxy-benzo[4,5]thieno[3,2-c]pyridine-3-carbonyl)-amino]-acetic acid,
[(1-Chloro-8-diethylamino-4-hydroxy-benzo[4,5]thieno[3,2-c]pyridine-3-carbonyl)-amino]-acetic acid,
[(8-Acetylamino-1-chloro-4-hydroxy-benzo[4,5]thieno[3,2-c]pyridine-3-carbonyl)-amino]-acetic acid,
[(4-Chloro-benzo[4,5]thieno[3,2-c]pyridine-3-carbonyl)-hydroxy-amino]-acetic acid,
[(1-Chloro-6-fluoro-4-hydroxy-benzo[4,5]thieno[3,2-c]pyridine-3-carbonyl)-amino]-acetic acid,
[(1-Chloro-7-fluoro-4-hydroxy-benzo[4,5]thieno[3,2-c]pyridine-3-carbonyl)-amino]-acetic acid,
[(1-Chloro-9-fluoro-4-hydroxy-benzo[4,5]thieno[3,2-c]pyridine-3-carbonyl)-amino]-acetic acid,
[(4-Hydroxy-1-pyridin-2-yl-1H-pyrazolo[3,4-c]pyridine-5-carbonyl)-amino]-acetic acid,
[(4-Hydroxy-1-methyl-benzo[4,5]thieno[3,2-c]pyridine-3-carbonyl)-amino]-acetic acid,
[Hydroxy-(4-hydroxy-benzo[4,5]thieno[3,2-c]pyridine-3-carbonyl)-amino]-acetic acid,
[(1-Chloro-4,8-dihydroxy-benzo[4,5]thieno[3,2-c]pyridine-3-carbonyl)-amino]-acetic acid,
[(1-Chloro-4-hydroxy-7-methoxy-benzo[4,5]thieno[3,2-c]pyridine-3-carbonyl)-amino]-acetic acid,
[(1-Chloro-8-hydroxy-4-methoxy-benzo[4,5]thieno[3,2-c]pyridine-3-carbonyl)-amino]-acetic acid,
[(1-Chloro-8-hydroxy-4-isopropoxy-benzo[4,5]thieno[3,2-c]pyridine-3-carbonyl)-amino]-acetic acid,
[(1-Chloro-4,7-dihydroxy-benzo[4,5]thieno[3,2-c]pyridine-3-carbonyl)-amino]-acetic acid,
[(1-Chloro-4-hydroxy-7-isopropoxy-benzo[4,5]thieno[3,2-c]pyridine-3-carbonyl)-amino]-acetic acid,
[(7-Fluoro-4-hydroxy-1-methyl-benzo[4,5]thieno[3,2-c]pyridine-3-carbonyl)-amino]-acetic acid,
[(1-Chloro-8-ethylamino-4-hydroxy-benzo[4,5]thieno[3,2-c]pyridine-3-carbonyl)-amino]-acetic acid,
[(8-Benzenesulfonylamino-1-chloro-4-hydroxy-benzo[4,5]thieno[3,2-c]pyridine-3-carbonyl)-amino]-acetic acid,
[(8-Benzylamino-1-chloro-4-hydroxy-benzo[4,5]thieno[3,2-c]pyridine-3-carbonyl)-amino]-acetic acid,
[(1-Chloro-4-hydroxy-8-trifluoromethyl-benzo[4,5]thieno[3,2-c]pyridine-3-carbonyl)-amino]-acetic acid,
[(1-Chloro-7-fluoro-4-hydroxy-2-oxy-benzo[4,5]thieno[3,2-c]pyridine-3-carbonyl)-amino]-acetic acid,
[(1-Chloro-4-hydroxy-8-phenylmethanesulfonyl-benzo[4,5]thieno[3,2-c]pyridine-3-carbonyl)-amino]-acetic acid,
[(1-Chloro-8-ethanesulfonyl-4-hydroxy-benzo[4,5]thieno[3,2-c]pyridine-3-carbonyl)-amino]-acetic acid,
[(8-Benzenesulfonyl-1-chloro-4-hydroxy-benzo[4,5]thieno[3,2-c]pyridine-3-carbonyl)-amino]-acetic acid,
[(8-Benzenesulfinyl-1-chloro-4-hydroxy-benzo[4,5]thieno[3,2-c]pyridine-3-carbonyl)-amino]-acetic acid, and
   or a pharmaceutically acceptable salt, ester, amide, or prodrug thereof.

In another aspect, the compounds disclosed herein have increased or decreased potency at HIF prolyl hydroxylases, bind the open or closed conformations of HIF pyrolyl hydroxylases, have more optimal pharmacokinetics, improved dosing schedules, less toxicity, have higher selectivity for HIF PH2 (less off-target activity), increase or decrease expression of HIF-regulated genes to a greater or lesser extent, or combinations of the preceding as compared to other HIF prolyl hydroxylase modulators.

### Synthesis

In another aspect, disclosed herein is a method of synthesizing the compounds of Formula I. Some of the compounds disclosed herein can be synthesized by using generally accepted organic synthetic methods, including the methodology shown in Scheme 1, below. Those of ordinary skill in the art recognize that some functional groups can be protected/deprotected using various protecting groups before a certain reaction takes place. The use of these protecting groups is well-known in the art, as for example set forth in Greene and Wuts, Protective Groups in Organic Synthesis, 3^{rd} Ed., John Wiley & Sons, New York, N.Y., 1999, which is incorporated herein in its entirety.

Various starting materials including aryl carboxylic acid derivatives may be prepared according to a variety of known synthetic methods. Some of these compounds are also commercially available from manufacturers and suppliers of reagents, such as Aldrich, Sigma, TCI, Wako, Kanto, Fluorchem, Acros, Abocado, Alfa, Fluka, etc.

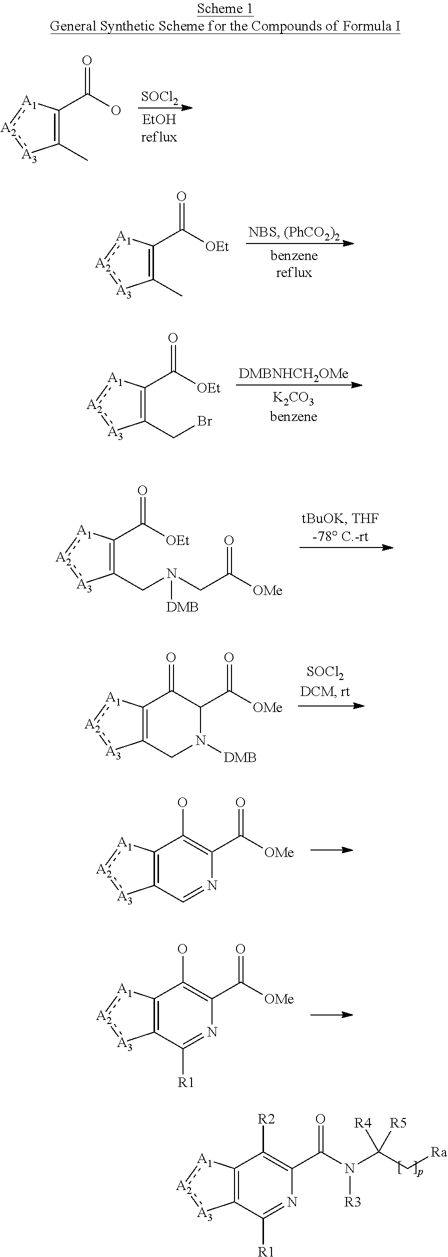

The compounds of Formula I may be synthesized from o-methyl-substituted aryl carboxylic acid according to the synthetic scheme shown Scheme 1. The carboxylic acid group is modified into esters by general esterification processes. The o-methyl group is brominated and is then condensed with a glycine ester having a DMB protective group. Cyclization and oxidation reactions, followed by a coupling reaction with various amines, result in the synthesis of the compounds of Formula I. The R₁ group can then be introduced through various substitution reactions after a radical halogenation step.

Another synthetic route that can be used to synthesize some of the compounds disclosed herein is shown in Scheme 2, below. Those of ordinary skill in the art recognize how to use the synthetic methodology shown in Scheme 2 to synthesize the other compounds disclosed herein.

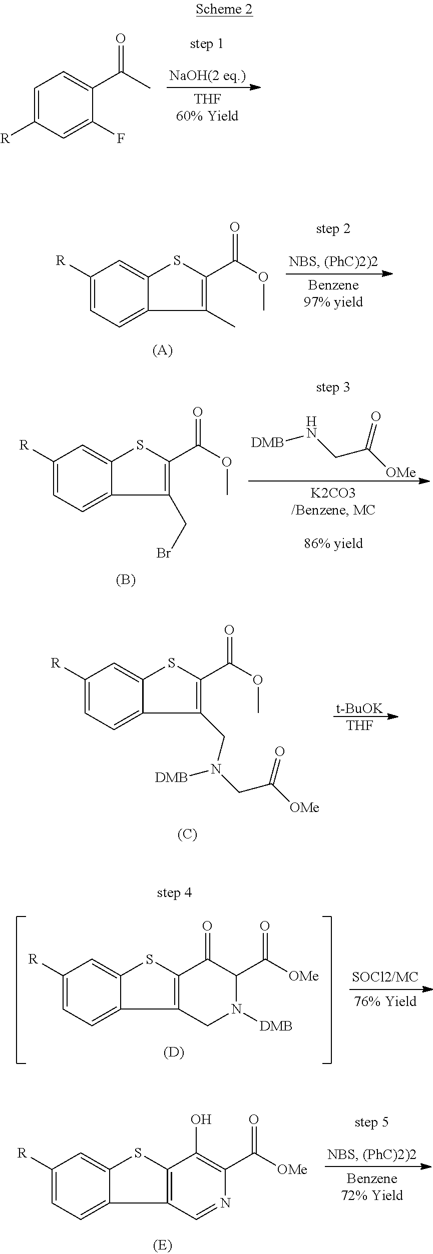

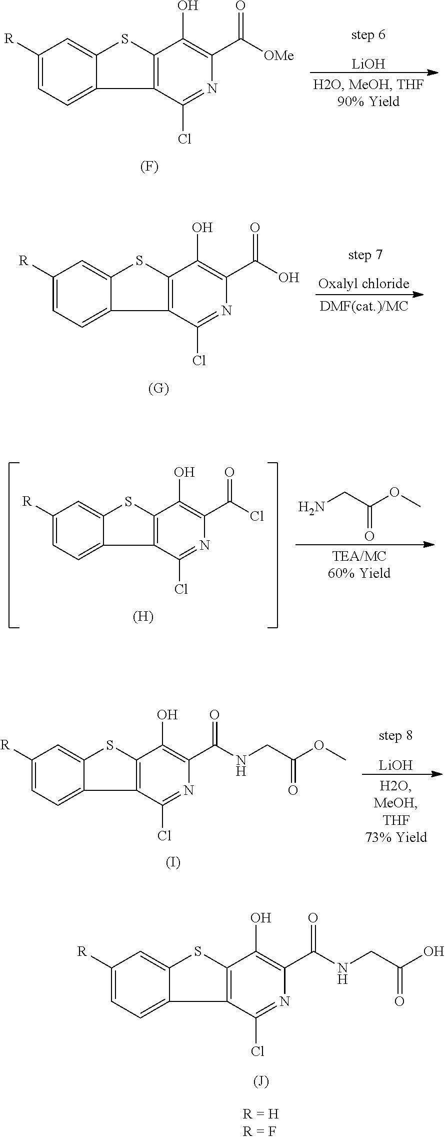

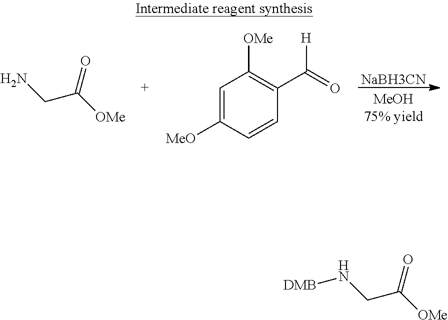

Intermediate (I) in Scheme 2 can be used as a starting point to synthesize some of the compounds disclosed herein, as shown in Scheme 3.

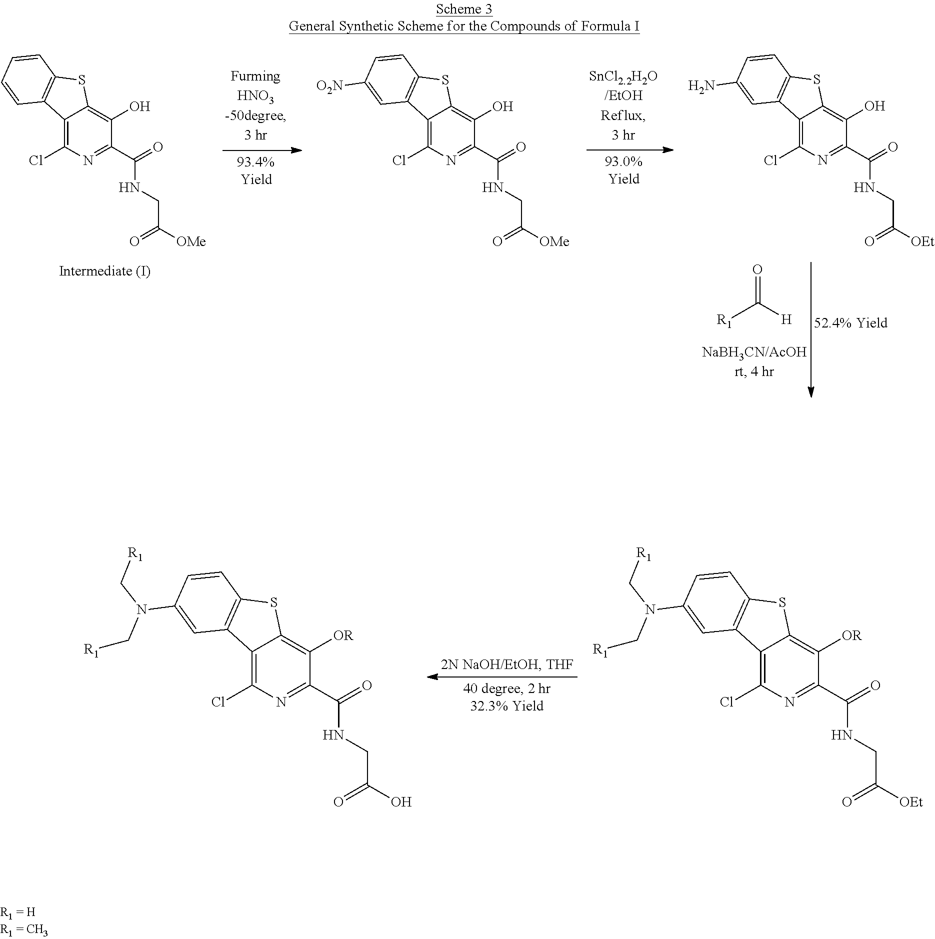

Another synthetic scheme that can be generalized and used to synthesize some of the compounds disclosed herein is shown in Scheme 4.

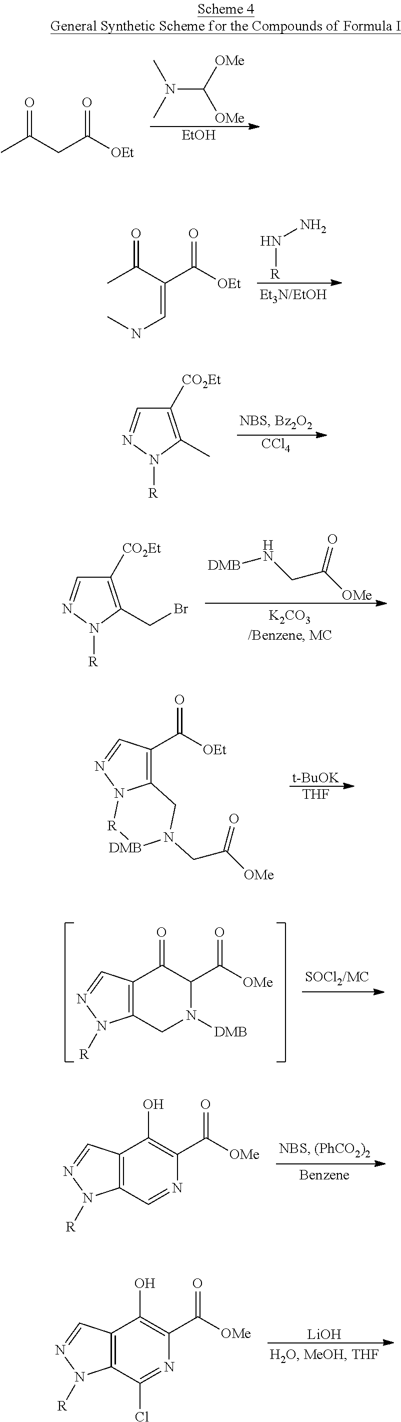

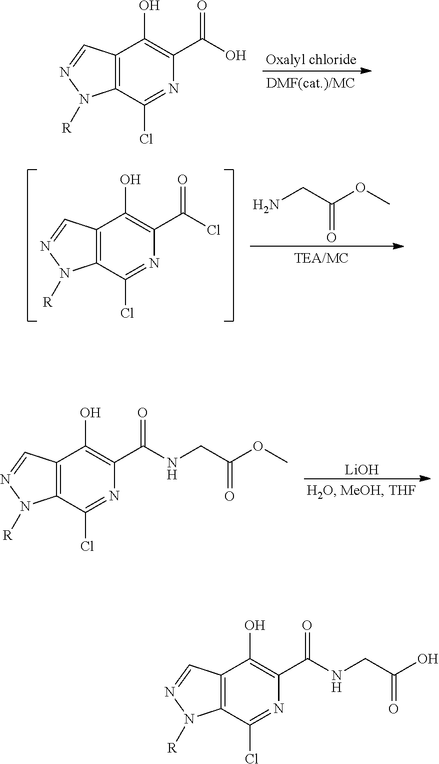

Some of the compounds disclosed herein can be synthesized according the procedure set forth in Scheme 5.

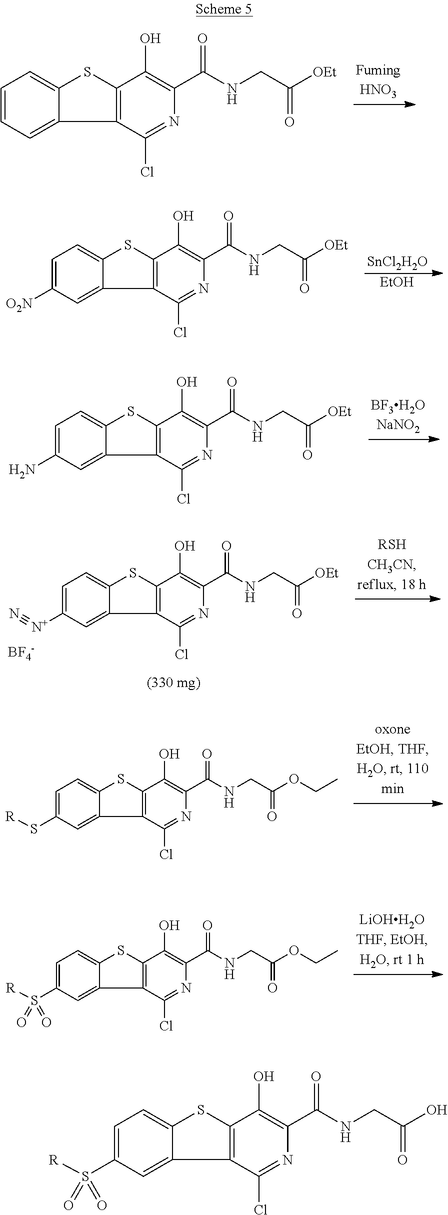

### Pharmaceutical Compositions

In another aspect, disclosed herein are pharmaceutical compositions comprising a therapeutically effective amount of at least one compound of Formula I-V and a physiologically acceptable carrier, diluent, or excipient.

The term “pharmaceutical composition” refers to a mixture of a compound disclosed herein with other chemical components, such as diluents or carriers. The pharmaceutical composition facilitates administration of the compound to a subject. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of a subject.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound and/or is not harmful to the subject to which it is administered.

The pharmaceutical compositions described herein can be administered to a subject per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternatively, one may administer the compound in a local rather than systemic manner, for example, via injection of the compound directly into the area of pain, often in a depot or sustained release formulation. Furthermore, one may administer the drug in a targeted drug delivery system, for example, in a liposome coated with a tissue-specific antibody. The liposomes will be targeted to and taken up selectively by the targeted organ.

The pharmaceutical compositions disclosed herein may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present disclosure thus may be formulated in a conventional mariner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may be used.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Ch. 1 p. 1). Typically, the dose about the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage. Where no human dosage is established, as will be the case for newly discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### Methods of Use

Throughout the present disclosure and the adjoining claims, the recitation of the term “compound of Formula I”, “compound of Formula II”, “compound of Formula III”, “compound of Formula IV”, “compound of Formula V”, or “compound of Formula I-V” includes in its scope those compounds as described herein, including any pharmaceutically acceptable salt, ester, amide, or prodrug thereof.

In another aspect, disclosed herein are methods of controlling the expression level of HIF in a cell, the method comprising administering to the cell an amount of at least one compound of Formula I-V sufficient to modulate the expression level of HIF in the cell. Similarly, disclosed herein are methods of controlling the expression level of HIF in a cell comprising contacting the cell with an amount of at least one compound of Formula I-V sufficient to modulate the expression level of HIF in the cell.

The term “administering” in the context of administering a compound refers to preparing a formulation, as discussed herein, containing the compound being administered, and administering the formulation by any known method to the subject or to the cell. For example, a solution containing the compound can be injected to the subject or be added to the medium containing the cells, or the subject can orally ingest a formulation containing the compound. The term “contacting” refers to bringing the subject or the cell into contact with the compound. Thus, a formulation of a prodrug can be administered to a subject, whereupon the prodrug undergoes metabolism. The metabolite is then either in the systemic circulation or within the cytoplasm. In this situation, the prodrug is “administered” to the subject, but both the subject and the cells are “contacted” with the metabolite.

In another aspect, disclosed herein are methods of controlling the expression level of HIF in a subject comprising identifying a subject in need thereof and administering to the subject an amount of at least one compound of Formula I-V sufficient to modulate the expression level of HIF in the subject. Similarly, disclosed herein are methods of controlling the expression level of HIF in a subject comprising identifying a subject in need thereof and contacting the subject with an amount of at least one compound of Formula I-V sufficient to modulate the expression level of HIF in the subject.

In another aspect, disclosed herein are methods for modulating the amount of HIF in a cell comprising administering to the cell, or contacting the cell with, an amount of at least one compound of Formula I-V sufficient to modulate the amount of HIF in the cell. Similarly, disclosed herein are methods for modulating the amount of HIF in a cell comprising administering to the cell, or contacting the cell with, an amount of at least one compound of Formula I-V sufficient to modulate the amount of HIF in the cell. The term “modulates” or “modulating” refers to the ability of a compound to alter the level or concentration of HIF. In some embodiments, the modulator increases the levels, or increases the concentration of HIF in the cell. In other embodiments, the modulator lowers the levels or concentration of HIF in the cell. Preferably, the modulator increases the levels or concentration of HIF in the cell.

In another aspect, disclosed herein are methods of inhibiting hydroxylation of HIFα in a cell comprising administering to the cell an amount of at least one compound of Formula I-V sufficient to inhibit the hydroxylation of HIFα in the cell. Similarly, disclosed herein are methods of inhibiting hydroxylation of HIFα in a cell comprising contacting the cell with an amount of at least one compound of Formula I-V sufficient to inhibit the hydroxylation of HIFα in the cell.

In another aspect, disclosed herein are methods of inhibiting hydroxylation of HIFα in a subject comprising identifying a subject in need thereof and administering to the subject an amount of at least one compound of Formula I-V sufficient to inhibit the hydroxylation of HIFα in the subject. Similarly, disclosed herein are methods of inhibiting hydroxylation of HIFα in a cell comprising identifying a subject in need thereof and contacting the subject with an amount of at least one compound of Formula I-V sufficient to inhibit the hydroxylation of HIFα in the subject.

In another aspect, disclosed herein are methods of modulating (increasing or decreasing) expression of HIF-regulated genes in a cell comprising administering to the cell an amount of at least one compound of Formula I-V sufficient to modulate expression of HIF-regulated genes in the cell. Similarly, disclosed herein are methods of modulate expression of HIF-regulated genes in a cell comprising contacting the cell with an amount of at least one compound of Formula I-V sufficient to modulate expression of HIF-regulated genes in the cell.

In another aspect, disclosed herein are methods of modulating expression of HIF-regulated genes in a subject comprising identifying a subject in need thereof and administering to the subject an amount of at least one compound of Formula I-V sufficient to modulate expression of HIF-regulated genes in the subject. Similarly, disclosed herein are methods of modulating expression of HIF-regulated genes in a subject comprising identifying a subject in need thereof and contacting the subject with an amount of at least one compound of Formula I-V sufficient to modulate expression of HIF-regulated genes in the subject.

In another aspect, disclosed herein are methods for increasing HIF levels or HIF activity in a cell comprising administering to the cell an amount of at least one compound of Formula I-V sufficient to increase HIF levels or HIF activity in the cell. Similarly, disclosed herein are methods for increasing HIF levels or HIF activity in a cell comprising contacting the cell with an amount of at least one compound of Formula I-V sufficient to increase HIF levels or HIF activity in the cell.

In another aspect, disclosed herein are methods for increasing HIF levels or HIF activity in a subject comprising identifying a subject in need thereof and administering to the subject an amount of at least one compound of Formula I-V sufficient to increase HIF levels or HIF activity in the subject. Similarly, disclosed herein are methods for increasing HIF levels or HIF activity in a subject comprising identifying a subject in need thereof and contacting the subject with an amount of at least one compound of Formula I-V sufficient to increase HIF levels or HIF activity in the subject.

In another aspect, disclosed herein are methods of treating a disorder in a subject where it is desired to modulate HIF levels or activity, the method comprising identifying a subject in need thereof and administering to the subject a therapeutically effective amount of at least one compound of Formula I-V. Similarly, disclosed herein are methods of treating an HIF-related disorder in a subject comprising identifying a subject in need thereof and contacting the subject with a therapeutically effective amount of at least one compound of Formula I-V. By HIF-related disorder is meant a disorder in which the modulation of HIF levels or activity provides a therapeutic effect.

In some embodiments, the HIF-related disorder is selected from the group consisting of ischemic disorders, hypoxic disorders, anemic disorders (including, but not limited to, anemia associated with autoimmune diseases, rheumatoid arthritis, systemic lupus, chronic infections such as, without limitation, HCV, and HIV, inflammatory bowel disease, chemotherapy-induced, chronic heart disease, chronic kidney disease, chronic obstructive pulmonary disease (COPD), end stage renal disease, prematurity, hypothyroidism, malnutrition, blood disorders, including but not limited to, sickle cell anemia, and β-thalassemia, malignancies), stenocardia, neurological disorders, stroke, epilepsy, neurodegenerative disease, myocardial infarction, liver ischemia, renal ischemia, chronic kidney disease, peripheral vascular disorders, ulcers, burns, chronic wounds, pulmonary embolism, ischemic-reperfusion injury, ischemic-reperfusion injuries associated with surgeries and organ transplantations, respiratory distress syndrome, prevention of broncho-pulmonary dysplasia in pre-maturity, pulmonary hypertension, auto-immune diseases, side effects of diabetes, diabetic retinopathy, macular degeneration, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndrome, toxoplasmosis, trauma and post-laser complications, diseases associated with rubeosis, metabolic disorders, for example diabetes, and proliferative vitreoretinopathy.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the patient's overall feeling of well being or appearance. Treatment may also include lengthening the life of the patient, even if the symptoms are not alleviated, the disease conditions are not ameliorated, or the patient's overall feeling of well being is not improved.

In another aspect, disclosed herein are methods of treating a disorder in a subject comprising identifying a subject in need thereof and administering to the subject a therapeutically effective amount of at least one compound of Formula I-V, wherein the disorder is selected from the group consisting of ischemic disorders, hypoxic disorders, anemic disorders (including, but not limited to, anemia associated with autoimmune diseases, rheumatoid arthritis, systemic lupus, chronic infections such as, without limitation, HCV, and HIV, inflammatory bowel disease, chemotherapy-induced, chronic heart disease, chronic kidney disease, chronic obstructive pulmonary disease. (COPD), end stage renal disease, prematurity, hypothyroidism, malnutrition, blood disorders, including but not limited to, sickle cell anemia, and p-thalassemia, malignancies), stenocardia, neurological disorders, stroke, epilepsy, neurodegenerative disease, myocardial infarction, liver ischemia, renal ischemia, chronic kidney disease, peripheral vascular disorders, ulcers, burns, chronic wounds, pulmonary embolism, ischemic-reperfusion injury, ischemic-reperfusion injuries associated with surgeries and organ transplantations, respiratory distress syndrome, prevention of broncho-pulmonary dysplasia in pre-maturity, pulmonary hypertension, auto-immune diseases, side effects of diabetes, diabetic retinopathy, macular degeneration, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndrome, toxoplasmosis, trauma and post-laser complications, diseases associated with rubeosis, metabolic disorders, for example diabetes, and proliferative vitreoretinopathy. Similarly, disclosed herein are methods of treating a disorder in a subject comprising identifying a subject in need thereof and contacting the subject with a therapeutically effective amount of at least one compound of Formula I-V, wherein the disorder is selected from the group consisting of anemic disorders, neurological disorders, stroke, trauma, epilepsy, neurodegenerative disease, myocardial infarction, liver ischemia, renal ischemia, peripheral vascular disorders, ulcers, burns, chronic wounds, pulmonary embolism, and ischemic-reperfusion injury.

In another aspect, disclosed herein are methods of inhibiting the activity of a hydroxylase enzyme which modifies the alpha subunit of HIF comprising contacting the enzyme with at least one compound of Formula I-V.

In another aspect, disclosed herein are methods of modulating the expression level of HIF and/or EPO by inhibiting the hydroxylation of HIFα, and thus stabilizing HIF and/or modulating expression of HIF-regulated genes. The method may be useful to prevent, remedy and treat conditions associated with HIF and/or EPO including anemia, ischemia and hypoxia.

Ischemia, anemia, and hypoxia are three conditions associated with HIF, and include, but are not limited to, of ischemic disorders, hypoxic disorders, anemic disorders (including, but not limited to, anemia associated with autoimmune diseases, rheumatoid arthritis, systemic lupus, chronic infections such as, without limitation, HCV, and HIV, inflammatory bowel disease, chemotherapy-induced, chronic heart disease, chronic kidney disease, chronic obstructive pulmonary disease (COPD), end stage renal disease, prematurity, hypothyroidism, malnutrition, blood disorders, including but not limited to, sickle cell anemia, and p-thalassemia, malignancies), stenocardia, neurological disorders, stroke, epilepsy, neurodegenerative disease, myocardial infarction, liver ischemia, renal ischemia, chronic kidney disease, peripheral vascular disorders, ulcers, burns, chronic wounds, pulmonary embolism, ischemic-reperfusion injury, ischemic-reperfusion injuries associated with surgeries and organ transplantations, respiratory distress syndrome, prevention of broncho-pulmonary dysplasia in pre-maturity, pulmonary hypertension, auto-immune diseases, side effects of diabetes, diabetic retinopathy, macular degeneration, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndrome, toxoplasmosis, trauma and post-laser complications, diseases associated with rubeosis, metabolic disorders, for example diabetes, and proliferative vitreoretinopathy. In some embodiments, the methods disclosed herein provide for stabilizing HIFα before/after the advent of the ischemia or hypoxia or in ischemia or hypoxia when the ischemia or hypoxia is associated with myocardial infarctions, strokes, or renal ischemia-reperfusion injuries.

In another aspect, disclosed herein are methods for treating a variety of ischemic- and/or hypoxic-related disorders using the compounds of Formula I-V. In certain embodiments, the methods disclosed herein are advantageous for the treatment when the compounds are administered before or after the advent of ischemia or hypoxia. For example, the methods disclosed herein may reduce mortality rates and improve cardiac structure and performance after the advent of the myocardial infarction.

Furthermore, disclosed herein are methods to treat liver disorders comprising administering the compounds of Formula I-V before or after exposure to conditions and/or agents that are associated with liver diesease. For example, hypoxia is associated with liver disease, particularly chronic liver disease that is associated with compounds toxic to the liver, such as ethanol. In addition, the expression of genes known to be regulated by HIFα, for example nitric oxide synthase and glucose transporter-1, is increased in alcoholic liver diseases.

Accordingly, disclosed herein are methods for treating conditions associated with ischemia or hypoxia, where the method includes administrating to subjects a therapeutically effective amount of at least one compound of Formula I-V.

In some embodiments, the compounds of Formula I-V are administered to patients after the onset of conditions such as acute ischemia, for example myocardial infarction, pulmonary embolism, bowel infarction, ischemic strokes, and renal ischemia-reperfusion injuries. In other embodiments, the compounds of Formula I-V are administered to patients after the patients are diagnosed with conditions associated with chronic ischemia, for example, without limitation, cardiachepatopathy, macular degeneration, pulmonary embolism, acute respiratory dysfunction, neonatal respiratory distress syndrome, and congestive heart failure. In other embodiments, the compounds of Formula I-V are administered to patients after trauma or injuries.

In another aspect, disclosed herein are methods for treating with the compounds disclosed herein patients at risk of developing ischemic or hypoxic conditions. High risk individuals, for example, include, but are not limited to, atherosclerotic patients. Risk factors in atherosclerosis include, for example without limitation, hyperlipidemia, smoking, hypertension, diabetes, hyperinsulinemia, and visceral obesity. Accordingly, disclosed herein are methods for preventing or mitigating ischemic tissue injuries, where the method includes administrating to subjects in need thereof a therapeutically effective amount of a compound of Formula I-V. In some embodiments, the compounds disclosed herein may be administered to treat conditions, such as, hypertension, diabetes, obliterative artery disease, chronic venous insufficiency, Raynaud's disease, chronic ulcer of skin, hepatopathy, congestive heart failure, and systemic sclerosis.

In some embodiments, the methods disclosed herein are used to stimulate angiogensis and/or formation of granulation tissue in injured tissues, and ulcers. For example, the compounds disclosed herein are effective in stimulating the formation of granulation tissue in the wound healing processes. Secretion of growth factors from inflammatory cells, blood platelets, and activated endothelia stimulates the translocation of fibroblast and endothelial cells and the growth in the granulation tissues. The methods disclosed herein are effective in stimulating the formation of granulation tissues. Accordingly, disclosed herein are methods for treating, for example, patients suffering from tissue injuries due to infarctions, patients suffering from injuries induced by trauma, or patients suffering from chronic injuries or ulcers caused by disorders, such as, diabetes. The methods disclosed herein include administering to subjects in need thereof a therapeutically effective amount of a compound of Formula I-V.

In another aspect, disclosed herein are methods for pre-treating subjects to reduce or prevent the development of tissue injuries associated with ischemia or hypoxia, by employing the compounds disclosed herein. The methods disclosed herein have advantages for the treatment when the compounds are administered before the advent of the ischemia or hypoxia. For example, the methods disclosed herein reduce mortality rates and significantly improve cardiac structure and performance when the compounds disclosed herein are administered before the induction of myocardial infarction. In addition, the methods disclosed herein provide a therapeutic effect associated with renal failure when the compounds disclosed herein are administered before and/or during the advent of ischemia-reperfusion injuries.

Accordingly, disclosed herein are methods for pre-treating subjects to reduce or prevent tissue injuries associated with ischemia or hypoxia, and the methods include administering a therapeutically effective amount of a compound disclosed herein to patients suffering from ischemic disorders, for example, those having a history of myocardial infarction, or patients suffering from symptoms of serious ischemia, for example stenocardia. In some embodiments, the compounds disclosed herein may be administered to humans who are under conditions that are associated with possible ischemia, for example general anesthesia, or who work temporarily at high altitudes. In other embodiments, the compounds disclosed herein may be used in organ transplant procedures by previously treating organ donors with the compounds disclosed herein to maintain the organs that have been removed from the donors before the organs are transplanted into recipients.

In another aspect, disclosed herein are methods for regulating angiogenesis in a subject comprising identifying a subject in need thereof and administering to the subject a therapeutically effective amount of at least one compound of Formula I-V. Similarly, disclosed herein are methods for regulating angiogenesis in a subject comprising identifying a subject in need thereof and contacting the subject with a therapeutically effective amount of at least one compound of Formula I-V.

In another aspect, disclosed herein are methods for vascularizing ischemic tissue in a subject comprising identifying a subject in need thereof and administering to the subject a therapeutically effective amount of at least one compound of Formula I-V. Similarly, disclosed herein are methods for vascularizing ischemic tissue in a subject comprising identifying a subject in need thereof and contacting the subject with a therapeutically effective amount of at least one compound of Formula I-V.

In another aspect, disclosed herein are methods for promoting the growth of skin graft replacements comprising identifying a subject in need thereof and administering to the subject a therapeutically effective amount of at least one compound of Formula I-V. Similarly, disclosed herein are methods for promoting the growth of skin graft replacements comprising identifying a subject in need thereof and contacting the subject with a therapeutically effective amount of at least one compound of Formula I-V.

In another aspect, disclosed herein are methods for promoting tissue repair in the context of guided tissue regeneration (GTR) procedures comprising identifying a subject in need thereof and administering to the subject a therapeutically effective amount of at least one compound of Formula I-V. Similarly, disclosed herein are methods for promoting tissue repair in the context of guided tissue regeneration (GTR) procedures comprising identifying a subject in need thereof and contacting the subject with a therapeutically effective amount of at least one compound of Formula I-V.

In another aspect, disclosed herein are methods for treating anemia in a subject comprising identifying a subject in need thereof and administering to the subject, or contacting the subject with, a therapeutically effective amount of at least one compound of Formula I-V. Similarly, disclosed herein are methods for treating anemia in a subject comprising identifying a subject in need thereof and administering to the subject, or contacting the subject with, a therapeutically effective amount of at least one compound of Formula I-V.

In another aspect, disclosed herein are methods for regulating anemia in a subject comprising identifying a subject in need thereof and administering to the subject a therapeutically effective amount of at least one compound of Formula I-V. Similarly, disclosed herein are methods for regulating anemia in a subject comprising identifying a subject in need thereof and contacting the subject with a therapeutically effective amount of at least one compound of Formula I-V.

In another aspect, disclosed herein are methods for preventing anemia in a subject comprising identifying a subject in need thereof and administering to the subject a therapeutically effective amount of at least one compound of Formula I-V. Similarly, disclosed herein are methods for preventing anemia in a subject comprising identifying a subject in need thereof and contacting the subject with a therapeutically effective amount of at least one compound of Formula I-V.

Disclosed herein are methods for increasing the level of endogenous erythropoietin (EPO). These methods may be used in vivo or in vitro, for example in cell culture-controlled media. In addition, disclosed herein are methods for increasing the level of endogenous EPO to prevent, remedy or treat conditions associated with deficient EPO levels or where increased EPO would be beneficial, such as in stroke patients, conditions associated with anemia and neurological disorders, e.g., Parkinson's disease. The conditions associated with decreased EPO levels include anemias, disorders such as acute or chronic renal diseases, diabetes, cancers, ulcers, acute or chronic infections, e.g., viral infections, such as HIV, bacterial infections, or parasitic infections; inflammatory disorders, autoimmune diseases, malignancies, severe trauma including thermal trauma, etc. These conditions are generally those that result in anemia in a subject. Furthermore, the methods disclosed herein are used to treat anemia associated with treatment procedures, such as radiation therapy, chemotherapy, dialysis, or surgery. Other examples of disorders associated with anemia include abnormal hemoglobin and/or hematocyte levels that are found in the disorders such as microcytic anemia, hypochromic anemia, aplastic anemia, etc.

The methods disclosed herein may be used to increase endogenous EPO levels in subjects undergoing prevention or certain treatment procedures. Examples include HIV-infected anemic subjects being treated with azidothymidine (zidovudin) or other reverse transcriptase inhibitors, patients receiving cyclic cisplatin- or non-cisplatin-containing chemotherapy, or anemic or non-anemic patients scheduled for surgical operations. The methods of increasing endogenous EPO levels may be used to prevent, pre-treat or treat EPO-related conditions that are associated with nerve injuries or degeneracy of nerve tissues, including, but not limited to, stroke, trauma, epilepsy, spinal cord injury, and neurodegenerative disorders.

In addition, the methods disclosed herein may be used to reduce the need for allogenic blood transfusions in anemic or non-anemic patients scheduled for surgery, such as joint replacement, or to facilitate autologous blood collection prior to surgery by increasing endogenous EPO levels. These methods would reduce the risk associated with non-autologous blood transfusions such as, without limitation, transmission of infectious disease.

The methods disclosed herein may also be used to enhance physical performance, improve exercise abilities, and facilitate or strengthen aerobic conditioning. These methods may, for example, be used for athletes to facilitate their training, and for military personnel to improve energy and stamina.

The methods disclosed herein may be used to increase endogenous erythropoietin levels in the blood serum of animals treated in media and in vivo from cells cultured in vitro. Although the kidney is a major in vivo source of erythropoietin, other organs including brain, liver and bone marrow may be made to produce erythropoietin when stimulated to do so. The methods disclosed herein may be used to increase the expression of endogenous erythropoietin in various organs including brain, kidney and liver.

The methods disclosed herein can be used to increase cell volume and hemoglobin level in animals that are treated in vivo with the compounds disclosed herein. The increase in plasma EPO, cell volume and hemoglobin levels in blood through the action of the compounds disclosed herein is sensitive to the amount of the compounds administered. It is therefore possible to establish a therapeutic regimen to produce a uniform and controlled level of the effect of the compounds disclosed herein.

The increase in cell volume and the hemoglobin in blood in the animals treated with the compounds disclosed herein causes an increase in the immature hematocytes (reticulocytes) circulating in the blood. Accordingly, disclosed herein are uses of the compounds disclosed herein for increasing reticulocyte levels in blood.

### EXAMPLES

### Example 1

### Synthesis of [(1-Chloro-4-hydroxy-benzo[4,5]thieno[3,2-c]pyridine-3-carbonyl)-amino]-acetic acid

### a) 3-Methyl-benzo[b]thiophene-2-carboxylic acid methyl ester

Thionyl chloride (15 g, 26 mmol) was added to methanol (100 ml), and 3-methyl-benzo[b]thiophene-2-carboxylic acid (5 g, 26.0 mmol) was dissolved, refluxed for 4 hours, and then evaporated to obtain a target compound 3-methyl-benzo[b]thiophene-2-carboxylic acid methyl ester (5.10 g, 24.7 mmol).

### b) 3-Bromomethyl-benzo[b]thiophene-2-carboxylic acid methyl ester

3-Methyl-benzo[b]thiophene-2-carboxylic acid methyl ester (0.200 g, 0.969 mmol) was dissolved in benzene, and NBS (0.173 g, 0.972 mmol) and a catalytic amount of benzoylperoxide was added, refluxed for 3 hours, cooled to room temperature, evaporated under a reduced pressure to remove solvents, and then purified with column chromatography to obtain a target compound 3-bromomethyl-benzo[b]thiophene-2-carboxylic acid methyl ester (0.248 g, 0.870 mmol).

### c) 3-{[(2,4-Dimethoxy-benzyl)-ethoxycarbonylmethyl-amino]-methyl}-benzo[b]thiophene-2-carboxylic acid methyl ester

3-Bromomethyl-benzo[b]thiophene-2-carboxylic acid methyl ester (1.60 g, 5.61 mmol) was dissolved in benzene, (2,4-Dimethoxy-benzylamino)-acetic acid methyl ester (1.56 g, 6.16 mmol) and potassium carbonate (0.853 g, 61.6 mmol) were added, stirred for 12 hours, diluted with ethyl acetate, washed with aq. NH₄Cl, dried, and then purified with silica gel column chromatography (eluant: n-Hex/EtOAC/DCM) to obtain a target compound (2.30 g, 5.19 mmol).

### d) 2-(2,4-Dimethoxy-benzyl)-4-oxo-1,2,3,4,4a,9b-hexahydro-benzo[4,5]thieno[3,2-c]pyridine-3-carboxylic acid ethyl ester

3-{[(2,4-Dimethoxy-benzyl)-ethoxycarbonylmethyl-amino]-methyl}-benzo[b]thiophene-2-carboxylic acid methyl ester (2.29 g, 5.16 mmol) was dissolved in anhydrous THF, and cooled in a dryice/acetone bath, and 1M potassium tert-butoxide THF solution (10.3 ml) was added by drop at the presence of nitrogen for 30 minutes, and then stirred at room temperature for 2 hours. 1N HCl was added to the resulting reaction solution, extracted with EtOAc, dried with MgSO₄, and then evaporated to obtain a target compound (1.12 g, 2.72 mmol).

### e) 4-Hydroxy-benzo[4,5]thieno[3,2-c]pyridine-3-carboxylic acid ethyl ester

2-(2,4-Dimethoxy-benzyl)-4-oxo-1,2,3,4,4a,9b-hexahydro-benzo[4,5]thieno[3,2-c]pyridine-3-carboxylic acid ethyl ester (0.917 g, 2.23 mmol) was dissolved in anhydrous dichloromethane (16 ml), and thionyl chloride (0.25 ml) was added, stirred at room temperature for 4 hours, neutralized with aq. sodium bicarbonate solution, and then extracted with dichloromethane. The resulting extract was dried with magnesium sulfate, and then purified with silica gel column chromatography (eluant: Hex/EtOAc) to obtain a target compound (0.974 g, 3.56 mmol).

### f) 1-Chloro-4-hydroxy-benzo[4,5]thieno[3,2-c]pyridine-3-carboxylic acid ethyl ester

4-Hydroxy-benzo[4,5]thieno[3,2-e]pyridine-3-carboxylic acid ethyl ester (0.380 g, 1.47 mmol) was dissolved in benzene, NCS (0.196 g, 1.47 mmol) and benzoylperoxide (35.5 mg, 0.147 mmol) were added, refluxed for 12 hours, evaporated under a reduced pressure to remove solvent off, and then purified with silica gel column chromatography (eluant Hex/EtOAc) to obtain a target compound (0.730 g, 2.49 mmol).

### g) [(1-Chloro-4-hydroxy-benzo[4,5]thieno[3,2-c]pyridine-3-carbonyl)-amino]-acetic acid

1-Chloro-4-hydroxy-benzo[4,5]thieno[3,2-c]pyridine-3-carboxylic acid ethyl ester (95.0 mg, 0.324 mmol) was dissolved in 0.5M sodium methoxide methanol solution (36.5 ml), reacted at 120° C. for 10 minutes in a microwave reactor, acidified with 1M HCl, and then purified with silica gel column chromatography (eluant: DCM/MeOH) to obtain a target compound (31.4 mg, 0.104 mmol).

### Example 2

### [(7-Hydroxy-3-phenyl-furo[3,2-c]pyridine-6-carbonyl)-amino]-acetic acid

### a) 4-Bromo-3-methyl-furan-2-carboxylic acid methyl ester

3-methyl-furan-2-carboxylic acid methyl ester (2.24 g, 16.0 mmol) was dissolved in acetonitrile (48 ml), and NBS (2.84 g, 16.0 mmol) was added, stirred at room temperature for 14 hours, and then purified with silica gel column chromatography (eluant: Hex/EtOAc) to obtain a target compound and a product mixture (1.53 g).

### b) 4-Bromo-3-bromomethyl-furan-2-carboxylic acid methyl ester

The previously prepared 4-bromo-3-methyl-furan-2-carboxylic acid methyl ester (1.53 g) was dissolved in benzene (21 ml), NBS (1.51 g, 8.50 mmol) and benzoylperoxide (0.206 g, 0.850 mmol) were added, refluxed for 15 hours, cooled to room temperature, evaporated under a reduced pressure to remove solvents, and then purified with column chromatography to obtain a mixture of 4-bromo-3-bromomethyl-furan-2-carboxylic acid methyl ester (1.96 g).

### c) 4-Bromo-3-{[(2,4-dimethoxy-benzyl)-methoxycarbonylmethyl-amino]-methyl}-furan-2-carboxylic acid methyl ester

The mixture of 4-bromo-3-bromomethyl-furan-2-carboxylic acid methyl ester (1.96 g) was dissolved in benzene (22 ml), and (2,4-Dimethoxy-benzylamino)-acetic acid methyl ester (2.13 g, 10.2 mmol) and potassium carbonate (1.45 g, 10.2 mmol) were added, stirred for 12 hours, diluted with ethyl acetate, washed with aq.NH₄Cl, dried, and then purified with silica gel column chromatography (eluant: n-Hex/EtOAC) to obtain target compounds 4-bromo-3-{[(2,4-dimethoxy-benzyl)-methoxycarbonylmethyl-amino]-methyl}-furan-2-carboxylic acid methyl ester (0.238 g, 0.522 mmol) and 3-{[(2,4-dimethoxy-benzyl)-methoxycarbonylmethyl-amino]-methyl}-furan-2-carboxylic acid methyl ester (1.25 g, 3.32 mmol).

### d) 3-{[2,4-Dimethoxy-benzyl)-methoxycarbonylmethyl-amino]-methyl}-4-phenyl-furan-2-carboxylic acid methyl ester

4-Bromo-3-{[(2,4-dimethoxy-benzyl)-methoxycarbonylmethyl-amino]-methyl}-furan-2-carboxylic acid methyl ester (0.160 g, 0.350 mmol), phenylboronic acid (85.2 mg, 0.699 mmol), Pd(dppf)Cl2 (17.1 mg, 21.0 umol), DPPF (11.6 mg, 21.0 umol), and potassium phosphate (81.6 mg, 0.385 mmol) were dissolved in DMF (2 ml), reacted at 100° C. for 15 hours, diluted with ethyl acetate, washed with brine, dried with magnesium sulfate, and then purified with silica gel column chromatography (eluant: Hex/EtOAc) to obtain 3-{[(2,4-dimethoxy-benzyl)-methoxycarbonylmethyl-amino]-methyl}-4-phenyl-furan-2-carboxylic acid methyl ester (0.103 g, 0.226 mmol) as a light yellow oil

### e) 5-(2,4-Dimethoxy-benzyl)-7-oxo-3-phenyl-4,5,6,7-tetrahydro-furo[3,2-c]pyridine-6-carboxylic acid methyl ester

3-{[(2,4-Dimethoxy-benzyl)-methoxycarbonylmethyl-amino]-methyl}-4-phenyl-furan-2-carboxylic acid methyl ester (0.103 g, 0.226 mmol) was dissolved in anhydrous THF (5 ml) and cooled in a dryice/acetone bath, and 1M potassium tert-butoxide THF solution (0.452 ml) was added by drop at the presence of nitrogen for 30 minutes, and then stirred at room temperature for 2 hours. 1N HCl was added to the resulting reaction solution, and the resulting mixture was extracted with EtOAc, dried with MgSO₄, evaporated to obtain a target compound (71.0 mg, 0.169 mmol).

### f) 7-Hydroxy-3-phenyl-furo[3,2-c]pyridine-6-carboxylic acid methyl ester

5-(2,4-Dimethoxy-benzyl)-7-oxo-3-phenyl-4,5,6,7-tetrahydro-furo[3,2-c]pyridine-6-carboxylic acid methyl ester (71.0 mg, 0.169 mmol) was dissolved in anhydrous dichloromethane, and thionyl chloride was added, stirred at room temperature for 3 hours, neutralized with aq. sodium bicarbonate solution, and then extracted with dichloromethane. The resulting extract was dried with magnesium sulfate, and then purified with silica gel column chromatography (eluant: Hex/EtOAc) to obtain a target compound (27.6 mg, 0.103 mmol).

### g) [(7-Hydroxy-3-phenyl-furo[3,2-c]pyridine-6-carbonyl)-amino]-acetic acid

7-Hydroxy-3-phenyl-furo[3,2-c]pyridine-6-carboxylic acid methyl ester (10.9 mg, 40.5 umol) and glycine (30.4 mg, 0.405 mmol) were dissolved in 0.5M sodium methoxide methanol solution, reacted at 120° C. for 10 minutes in a CEM microwave reactor, oxidified with 1M HCl, and then purified with silica gel column chromatography (eluant: DCM/MeOH) to obtain a target compound (1.27 mg, 4.07 umol).

### Example 3

### Analytical Data

Analytical data of the final target compounds that were synthesized using the above-mentioned methods are listed, as follows.

### [(4-Hydroxy-benzo[4,5]furo[3,2-c]pyridine-3-carbonyl)-amino]-acetic acid

¹H NMR (300 MHz, DMSO-d₆) δ 9.41 (s, 1H), 8.96 (s, 1H), 8.27 (bs, 1H), 7.82 (bs, 1H), 7.62 (d, 1H, J=6.9 Hz), 7.49 (bs, 1H), 4.00 (s, 2H). m/z=286.9 (M+H)

### [(4-Hydroxy-benzo[4,5]thieno[3,2-c]pyridine-3-carbonyl)-amino]-acetic acid

¹H NMR (300 MHz, DMSO-d₆) δ 9.43 (s, 1H), 9.22 (s, 1H), 8.59 (bs, 1H), 8.192 (bs, 1H), 7.63 (bs, 2H), 4.03 (s, 2H). m/z=303.0 (M+H)

### [(1-Chloro-4-hydroxy-benzo[4,5]thieno[3,2-c]pyridine-3-carbonyl)-amino]-acetic acid

1H NMR (300 MHz, CD₃OD) δ 8.96 (m, 1H), 8.09 (m, 1H), 7.65 (m, 2H), 4.12 (s, 2H)

### [(7-Hydroxy-3-phenyl-furo[3,2-c]pyridine-6-carbonyl)-amino]-acetic acid

¹H NMR (300 MHz, DMSO-d₆) δ 9.34 (s, 1H), 8.97 (s, 1H), 8.71 (bs, 1H), 8.46 (bs, 1H), 7.95 (bs, 2H), 7.59-7.54 (m, 3H), 3.77 (s, 2H). m/z=313.1 (M+H)

### [(4-Hydroxy-benzo[4,5]furo[3,2-c]pyridine-3-carbonyl)-amino]-acetic acid

¹H NMR (300 MHz, DMSO-d₆) δ 9.41 (s, 1H), 8.96 (s, 1H), 8.27 (bs, 1H), 7.82 (bs, 1H), 7.62 (d, 1H, J=6.9 Hz), 7.49 (bs, 1H), 4.00 (s, 2H). m/z=287 (M+H)

### [(4-Hydroxy-benzo[4,5]thieno[3,2-c]pyridine-3-carbonyl)-amino]-acetic acid

¹H NMR (300 MHz, DMSO-d₆) δ 9.43 (s, 1H), 9.22 (s, 1H), 8.59 (bs, 1H), 8.192 (bs, 1H), 7.63 (bs, 2H), 4.03 (s, 2H). m/z=303 (M+H)

### [(1-Chloro-4-hydroxy-benzo[4,5]thieno[3,2-c]pyridine-3-carbonyl)-amino]acetic acid

¹H NMR (300 MHz, DMSO-d₆) δ 8.96 (m, 1H), 8.09 (m, 1H), 7.65 (m, 2H), 4.12 (s, 2H). m/z=338 (M+H)

### [(7-Hydroxy-furo[3,2-c]pyridine-6-carbonyl)-amino]-acetic acid

¹H NMR (300 MHz, CD₃OD) δ 8.37 (s, 1H), 8.02 (s, 1H), 7.05 (s, 1H), 4.14 (s, 1H). m/z=237 (M+H)

### [(7-Hydroxy-2-phenyl-furo[3,2-c]pyridine-6-carbonyl)-amino]-acetic acid

¹H NMR (300 MHz, DMSO-d₆) δ 9.34 (s, 1H), 8.97 (s, 1H), 8.71 (bs, 1H), 8.46 (bs, 1H), 7.95 (bs, 2H), 7.59-7.54 (m, 3H), 3.77 (s, 2H). m/z=313 (M+H)

### (S)-2-[(7-Hydroxy-furo[3,2-c]pyridine-6-carbonyl)-amino]-propionic acid

¹H NMR (300 MHz, DMSO-d₆) δ 9.19 (broad, 1H), 8.54 (m, 1H), 8.25 (m, 1H), 7.20 (m, 1H), 4.46 (m, 1H), 1.45 (d, 3H). m/z=251 (M+H)

### [(4-Hydroxy-1-phenyl-1H-pyrazolo[3,4-c]pyridine-5-carbonyl)-amino]-acetic acid

¹H NMR (300 MHz, CD₃OD) δ 8.66 (s, 1H), 8.46 (s, 1H), 7.79 (d, J=6.9 Hz, 2H), 7.63 (t, J=6.9 Hz, 2H), 7.49 (t, J=6.9 Hz, 1H), 4.16 (s, 2H). m/z=313 (M+H)

### [(7-Chloro-4-hydroxy-1-phenyl-1H-pyrazolo[3,4-c]pyridine-5-carbonyl)-amino]-acetic acid

¹H NMR (300 MHz, CD₃OD) δ 8.50 (s, 1H), 7.61-7.48 (m, 5H), 4.14 (s, 2H). m/z=348 (M+H)

### [(1-Chloro-4-hydroxy-8-nitro-benzo[4,5]thieno[3,2-c]pyridine-3-carbonyl)-amino]-acetic acid

¹H NMR (300 MHz, DMSO-d₆) δ 9.55 (s, 1H), 9.40 (s, 1H), 8.52 (m, 2H), 4.03 (d, 2H, J=6.0). m/z=383 (M+H)

### 3-(Carboxymethyl-carbamoyl)-1-chloro-4-hydroxy-benzo[4,5]thieno[3,2-c]pyridin-8-yl-ammonium

¹H NMR (300 MHz, DMSO-d₆) δ 9.41 (br s, 2H), 8.33 (s, 1H), 8.00 (d, 1H, J=8.7), 7.21 (q, 1H), 4.11 (d, 2H, J=5.7). m/z=353 (M+H)

### [(1-Bromo-4-hydroxy-benzo[4,5]thieno[3,2-c]pyridine-3-carbonyl)-amino]-acetic acid

¹H NMR (300 MHz, DMSO-d₆) δ 9.36 (s, 1H), 9.12-9.09 (m, 1H), 8.30-8.27 (m, 1H), 7.77-7.69 (m, 2H), 4.03 (d, J=6.0 Hz, 2H). m/z=382 (M+H)

### (S)-2-[(1-Chloro-4-hydroxy-benzo[4,5]thieno[3,2-c]pyridine-3-carbonyl)-amino]-propionic acid

¹H NMR (300 MHz, CD₃OD) δ 8.97 (m, 1H), 8.10 (m, 1H), 7.69-7.65 (m, 2H), 4.66 (q, 1H), 1.59 (d, 3H). m/z=352 (M+H)

### (S)-2-[(1-Bromo-4-hydroxy-benzo[4,5]thieno[3,2-c]pyridine-3-carbonyl)-amino]-propionic acid

¹H NMR (300 MHz, CD₃OD) δ 9.12 (d, 1H), 8.05 (d, 1H), 7.67-7.62 (m, 2H), 4.67 (q, 1H), 1.60 (d, 3H). m/z=396 (M+H)

### [(1-Chloro-8-fluoro-4-hydroxy-benzo[4,5]thieno[3,2-c]pyridine-3-carbonyl)-amino]-acetic acid

¹H NMR (300 MHz, DMSO-d₆) δ 9.76 (s, 1H), 8.53 (d, 1H, J=9.3), 8.26 (m, 1H), 7.60 (m, 1H), 3.97 (d, 2H, J=4.5). m/z=356 (M+H)

### [(1-Cyano-4-hydroxy-benzo[4,5]thieno[3,2-c]pyridine-3-carbonyl)-amino]-acetic acid

¹H NMR (300 MHz, DMSO-d₆) δ 9.65 (s, 1H), 8.80 (s, 1H), 8.396 (s, 1H), 7.83 (m, 2H), 4.07 (s, 2H). m/z=328 (M+H)

### [1-Benzenesulfonyl-7-chloro-4-hydroxy-1H-pyrrolo[2,3-c]pyridine-5-carbonyl)-amino]-acetic acid

¹H NMR (300 MHz, DMSO-d₆) δ 9.24 (broad, 1H), 8.16 (m, 1H), 7.92 (d, 2H, J=7.5), 7.77 (m, 1H), 7.64 (m, 2H), 7.09 (d, 1H, J=3.6), 3.83 (d, 2H, J=4.8). m/z=411 (M+H)

### [(1-Benzenesulfonyl-7-chloro-4-hydroxy-1H-pyrrolo[2,3-c]pyridine-5-carbonyl)-amino]-acetic acid methyl ester

¹H NMR (300 MHz, CDCl₃) δ 12.22 (s, 1H), 8.04 (d, 2H, J=3.9), 7.83 (d, 2H, J=5.7), 7.64 (m, 1H), 7.52 (m, 2H), 7.01 (d, 1H, J=3.9), 4.21 (d, 2H, J=5.7), 3.77 (s, 3H). m/z=425 (M+H)

### [(7-Chloro-4-hydroxy-1H-pyrrolo[2,3-c]pyridine-5-carbonyl)-amino]-acetic acid

¹H NMR (300 MHz, DMSO-d₆) δ 8.52 (s, 1H), 7.59 (m, 1H), 7.25 (m, 2H), 2.90 (d, 2H). m/z=271 (M+H)

### [(4-Amino-1-bromo-benzo[4,5]thieno[3,2-c]pyridine-3-carbonyl)-amino]-acetic acid

¹H NMR (300 MHz, DMSO-d₆) δ 9.16 (d, J=7.8 Hz, 1H), 8.06 (d, J=7.8 Hz, 1H), 7.65-7.63 (m, 2H), 4.14 (s, 2H). m/z=381 (M+H)

### 1-Bromo-4-hydroxy-benzo[4,5]thieno[3,2-c]pyridine-3-carboxylic acid (pyridin-3-ylmethyl)-amide

¹H NMR (300 MHz, DMSO-d₆) δ 9.88 (s, 1H), 9.10-9.07 (m, 1H), 8.60 (s, 1H), 8.46 (s, 1H), 8.27-8.24 (m, 1H), 7.79-7.67 (m, 3H), 7.38-7.34 (m, 1H), 4.57 (d, J=6.3 Hz, 2H). m/z=415 (M+H)

### [(1-Bromo-4-fluoro-benzo[4,5]thieno[3,2-c]pyridine-3-carbonyl)-amino]-acetic acid

¹H NMR (300 MHz, DMSO-d₆) δ 9.07 (d, 1H, J=8.7), 8.88 (s, 1H), 8.24 (d, 1H, J=8.4), 7.72 (m, 2H), 3.96 (d, 2H, J=6.0). m/z=384 (M+H)

### 1-Bromo-4-hydroxy-benzo[4,5]thieno[3,2-c]pyridine-3-carboxylic acid [244-methoxy-benzyl)-2H-tetrazol-5-ylmethyl]-amide

¹H NMR (300 MHz, CDCl₃) δ 12.40 (s, 1H), 8.41 (s, 1H), 7.95 (s, 1H), 7.65-7.56 (m, 2H0, 7.36 (d, J=8.7 Hz, 2H), 6.89 (d, J=8.7 Hz, 2H), 5.70 (s, 2H), 4.94 (d, J=6 Hz, 2H), 3.79 (s, 3H). m/z=526 (M+H)

### 1-Bromo-4-hydroxy-benzo[4,5]thieno[3,2-c]pyridine-3-carboxylic acid[1-(4-methoxy-benzyl)-1H-tetrazol-5-ylmethyl]-amide

¹H NMR (300 MHz, CDCl₃) δ 12.04 (s, 1H), 9.19-9.15 (m, 1H), 8.26 (s, 1H), 8.01-7.98 (m, 1H), 7.67-7.63 (m, 2H), 7.15 (d, J=8.7 Hz, 2H), 6.76 (d, J=8.7 Hz, 2H), 5.68 (s, 2H), 4.91 (d, J=6.3 Hz, 2H), 3.58 (s, 3H). m/z=526 (M+H)

### 1-Bromo-4-hydroxy-benzo[4,5]thieno[3,2-c]pyridine-3-carboxylic acid (pyridin-2-ylmethyl)-amide

¹H NMR (300 MHz, CDCl₃) δ 9.20-9.17 (m, 1H), 8.85 (s, 1H), 8.66 (s, 1H), 8.00-7.96 (m, 1H), 7.90 (s, 1H), 7.41 (s, 1H), 4.95 (s, 2H). m/z=415 (M+H)

### 1-Bromo-4-hydroxy-benzo[4,5]thieno[3,2-c]pyridine-3-carboxylic acid (1H-tetrazol-5-ylmethyl)-amide

¹H NMR (300 MHz, DMSO-d₆) δ 9.84 (s, 1H), 9.11 (d, J=7.2 Hz, 1H), 8.29 (d, J=7.2 Hz, 1H), 7.77-7.71 (m, 2H), 4.85 (d, J=5.7 Hz, 2H). m/z=406 (M+H)

### 1-Bromo-4-hydroxy-benzo[4,5]thieno[3,2-c]pyridine-3-carboxylic acid pyridin-2-ylamide

¹H NMR (300 MHz, DMSO-d₆) δ 9.12 (d, J=8.7 Hz, 1H), 8.44 (d, J=4.2 Hz, 1H), 8.30 (d, J=6.6 Hz, 1H), 8.18 (d, J=7.8 Hz, 1H), 7.94 (t, J=7.5 Hz, 1H), 7.77-7.74 (m, 2H), 7.28-7.24 (m, 1H). m/z=401 (M+H)

### 1-Bromo-4-hydroxy-benzo[4,5]thieno[3,2-c]pyridine-3-carboxylic acid pyridin-3-ylamide

¹H NMR (300 MHz, DMSO-d₆) δ 9.09 (m, 1H), 8.92 (s, 1H), 8.32 (s, 1H), 8.23-8.20 (m, 2H), 7.69-7.66 (m, 2H), 7.43-7.38 (m, 1H). m/z=401 (M+H)

### 1-Bromo-4-hydroxy-benzo[4,5]thieno[3,2-c]pyridine-3-carboxylic acid phenylamide

¹H NMR (300 MHz, DMSO-d₆) δ 10.85 (s, 1H), 9.14 (d, J=8.7 Hz, 1H), 8.30 (d, J=8.7 Hz, 1H), 7.83 (d, J=7.8 Hz, 2H), 7.76-7.74 (m, 2H), 7.42 (t, J=7.5 Hz, 2H), 7.21 (t, J=7.2 Hz, 1H). m/z=400 (M+H)

### 1-Bromo-4-hydroxy-benzo[4,5]thieno[3,2-c]pyridine-3-carboxylic acid benzylamide

¹H NMR (300 MHz, CDCl₃) δ 9.18-9.15 (m, 1H), 8.21 (s, 1H), 8.00-7.97 (m, 1H), 7.66-7.61 (m, 2H), 7.41-7.33 (m, 5H), 4.70 (d, J=6.3 Hz, 2H). m/z=414 (M+H)

### [(1-Chloro-8-dimethylamino-4-hydroxy-benzo[4,5]thieno[3,2-c]pyridine-3-carbonyl)-amino]-acetic acid

1H NMR (300 MHz, DMSO-d₆) δ 8.31 (s, 1H), 8.18 (s, 1H), 8.00 (d, 1H), 7.28 (d, 1H), 3.99 (s, 2H), 3.04 (s, 6H). m/z=381 (M+H)

### [(1-Chloro-8-diethylamino-4-hydroxy-benzo[4,5]thieno[3,2-c]pyridine-3-carbonyl)-amino]-acetic acid

¹H NMR (300 MHz, DMSO-d₆) δ 8.31 (s, 1H), 8.15 (s, 1H), 7.75 (d, 1H), 7.11 (m, 1H), 4.08 (s, 2H), 3.60-3.42 (m, 4H), 1.26-1.17 (m, 6H). m/z=409 (M+H)

### [(8-Acetylamino-1-chloro-4-hydroxy-benzo[4,5]thieno[3,2-c]pyridine-3-carbonyl)-amino]-acetic acid

¹H NMR (300 MHz, DMSO-d₆) δ 12.98 (br, 1H), 10.33 (s, 1H), 9.37 (s, 2H), 8.18 (d, 1H), 7.90 (d, 1H), 4.04 (d, 2H), 2.13 (s, 3H). m/z=395 (M+H)

### [(4-Chloro-benzo[4,5]thieno[3,2-c]pyridine-3-carbonyl)-hydroxy-amino]-acetic acid

¹H NMR (300 MHz, DMSO-d₆) δ 11.80 (s, 1H), 9.26 (m, 1H), 8.98 (m, 2H), 8.40 (m, 2H), 8.28 (m, 1H), 3.80 (m, 2H). m/z=338 (M+H)

### [(1-Chloro-6-fluoro-4-hydroxy-benzo[4,5]thieno[3,2-c]pyridine-3-carbonyl)-amino]-acetic acid

¹H NMR (300 MHz, CD₃OD) δ 8.78 (d, 1H), 7.64 (m, 1H), 7.44 (t, 1H), 4.12 (s, 2H). m/z=356 (M+H)

### [(1-Chloro-7-fluoro-4-hydroxy-benzo[4,5]thieno[3,2-c]pyridine-3-carbonyl)-amino]-acetic acid

¹H NMR (300 MHz, CDCl₃) δ 13.09 (s, 1H), 9.33 (t, 1H, J=6.3 Hz), 8.39 (dd, 1H, J=2.4 Hz, 9.0 Hz), 8.25 (dd, 1H, J=2.1 Hz, 9.3 Hz), 7.59 (m, 1H) 4.01 (s, 2H). m/z=356 (M+H)

### [(1-Chloro-9-fluoro-4-hydroxy-benzo[4,5]thieno[3,2-c]pyridine-3-carbonyl)-amino]-acetic acid

¹H NMR (300 MHz, DMSO-d₆) δ 12.91 (s, 1H), 9.33 (t, J=6.0 Hz, 1H), 8.10 (d, J=8.1 Hz, 1H), 7.78-7.71 (m, 1H), 7.51-7.44 (m, 1H), 4.02 (d, J=6 Hz, 2H). m/z=356 (M+H)

### [(4-Hydroxy-1-pyridin-2-yl-1H-pyrazolo[3,4-c]pyridine-5-carbonyl)-amino]-acetic acid

¹H NMR (300 MHz, DMSO-d₆) δ 9.55 (s, 1H), 9.27 (s, 1H), 8.73 (s, 1H), 8.64 (d, J=4.8 Hz, 1H), 8.11-8.04 (m, 2H), 7.46-7.42 (m, 1H), 4.01 (d, J=5.7 Hz, 2H). m/z=314 (M+H)

### [(4-Hydroxy-1-methyl-benzo[4,5]thieno[3,2-c]pyridine-3-carbonyl)-amino]-acetic acid

¹H NMR (300 MHz, DMSO-d₆) δ 9.30 (s, 1H), 8.48 (s, 1H), 8.23 (s, 1H), 7.66 (s, 2H), 4.01 (d, J=4.8 Hz, 2H), 3.04 (s, 3H). m/z=317 (M+H)

### [Hydroxy-(4-hydroxy-benzo[4,5]thieno[3,2-c]pyridine-3-carbonyl)-amino]-acetic acid

¹H NMR (300 MHz, DMSO-d₆) δ 13.42 (s, 1H), 8.73 (s, 1H), 8.51 (d, J=6.9 Hz, 1H), 7.90 (d, J=6.9 Hz, 1H), 7.56-7.47 (m, 2H), 4.49 (s, 2H). m/z=319 (M+H)

[(1-Chloro-4,8-dihydroxy-benzo[4,5]thieno[3,2-c]pyridine-3-carbonyl)-amino]-acetic acid

¹H NMR (300 MHz, DMSO-d₆) δ 10.03 (s, 1H), 9.38 (s, 1H), 8.28 (d, J=1.8 Hz, 1H), 8.03 (d, J=8.7 Hz, 1H), 7.19 (dd, J=8.7 Hz and 2.4 Hz, 1H), 4.01 (d, J=6.3 Hz, 2H). m/z=354 (M+H)

### [(1-Chloro-4-hydroxy-7-methoxy-benzo[4,5]thieno[3,2-c]pyridine-3-carbonyl)-amino]-acetic acid

¹H NMR (300 MHz, DMSO-d₆) δ 13.03 (s, 1H), 12.78 (s, 1H), 9.24 (s, 1H), 8.75 (d, J=9 Hz, 1H), 7.87 (d, J=2.1 Hz, 1H), 7.28 (dd, J=9.3 Hz and 2.1 Hz, 1H), 4.02 (d, J=5.7 Hz, 2H) 3.90 (s, 3H). m/z=368 (M+H)

### [(1-Chloro-8-hydroxy-4-methoxy-benzo[4,5]thieno[3,2-c]pyridine-3-carbonyl)-amino]-acetic acid

¹H NMR (300 MHz, CD₃OD) δ 8.35 (d, J=2.1 Hz, 1H), 7.86 (d, J=8.7 Hz, 1H), 7.18 (dd, J=2.1 Hz and 0.9 Hz, 1H), 4.17 (s, 2H), 4.11 (s, 3H). m/z=368 (M+H) [(1-Chloro-8-hydroxy-4-isopropoxy-benzo[4,5]thieno[3,2-c]pyridine-3-carbonyl)-amino]-acetic acid

¹H NMR (300 MHz, CD₃OD) δ 8.36 (d, J=2.4 Hz, 1H), 7.85 (d, J=8.7 Hz, 1H), 7.18 (dd, J=8.4 Hz and 2.4 Hz, 1H), 4.12 (s, 2H), 1.38 (s, 3H), 1.36 (s, 3H). m/z=396 (M+H)

### [(1-Chloro-4,7-dihydroxy-benzo[4,5]thieno[3,2-c]pyridine-3-carbonyl)-amino]-acetic acid

¹H NMR (300 MHz, DMSO-d₆) δ 12.9-12.6 (br s, 1H), 10.42 (s, 1H), 9.31 (s, 1H), 8.68 (d, J=9.0 Hz, 1H), 7.54 (s, 1H), 7.14 (dd, J=8.7 Hz and 2.4 Hz, 1H), 4.00 (d, J=6.3 Hz, 1H). m/z=354 (M+H)

### [(1-Chloro-4-hydroxy-7-isopropoxy-benzo[4,5]thieno[3,2-c]pyridine-3-carbonyl)-amino]-acetic acid

¹H NMR (300 MHz, DMSO-d₆) δ 13.2-12.8 (br s, 2H), 9.40 (s, 1H), 8.72 (d, J=9 Hz, 1H), 7.86 (s, 1H), 7.25 (dd, J=9.3 Hz and 2.1 Hz, 1H), 4.82-4.78 (m, 1H), 4.00 (d, J=6 Hz, 2H), 1.34 (s, 3H), 1.32 (s, 3H). m/z=396 (M+H)

### [(7-Fluoro-4-hydroxy-1-methyl-benzo[4,5]thieno[3,2-c]pyridine-3-carbonyl)-amino]-acetic acid

¹H NMR (300 MHz, DMSO-d₆) δ 9.26 (s, 1H), 8.49 (s, 1H), 8.19 (d, J=7.5 Hz, 1H), 7.53 (m, 1H), 3.96 (s, 1H), 3.02 (s, 3H). m/z=335 (M+H)

### [(1-Chloro-8-ethylamino-4-hydroxy-benzo[4,5]thieno[3,2-c]pyridine-3-carbonyl)-amino]-acetic acid

¹H NMR (300 MHz, DMSO-d₆) δ 13.01 (s, 1H), 9.25 (t, J=6.0 Hz, 1H), 7.99 (d, J=2.1 Hz, 1H), 7.90 (d, J=9.0 Hz, 1H), 7.06 (m, 1H), 4.02 (d, J=6.3 Hz, 2H), 3.15 (q, J=7.2 Hz, 2H), 1.23 (t, J=7.2 Hz, 3H). m/z=381 (M+H)

### [(8-Benzenesulfonylamino-1-chloro-4-hydroxy-benzo[4,5]thieno[3,2-c]pyridine-3-carbonyl)-amino]-acetic acid

¹H NMR (300 MHz, DMSO-d₆) δ 9.43-9.26 (m, 1H), 8.70 (s, 1H), 8.14 (d, J=8.7, 1H), 7.83 (d, J=8.2, 2H), 7.55 (d, J=7.3, 3H), 7.43 (d, J=8.7, 1H), 4.02 (d, J=6.1, 2H). m/z=493 (M+H)

### [(8-Benzylamino-1-chloro-4-hydroxy-benzo[4,5]thieno[3,2-c]pyridine-3-carbonyl)-amino]-acetic acid

¹H NMR (300 MHz, DMSO-d₆) δ 9.42-9.25 (m, 1H), 8.01-7.80 (m, 3H), 7.49-7.17 (m, 5H), 7.09 (d, J=8.7, 1H), 4.39 (d, J=5.6, 2H), 3.99 (d, J=6.0, 2H). m/z=443 (M+H)

### [(1-Chloro-4-hydroxy-8-trifluoromethyl-benzo[4,5]thieno[3,2-c]pyridine-3-carbonyl)-amino]-acetic acid

¹H NMR (300 MHz, DMSO-d₆) δ 9.54 (brs, 1H), 9.11 (s, 1H), 8.56 (d, J=8.4 Hz, 1H), 8.07 (d, J=8.4 Hz, 1H), 4.02 (d, J=6 Hz, 2H). m/z=406 (M+H)

### [(1-Chloro-7-fluoro-4-hydroxy-2-oxy-benzo[4,5]thieno[3,2-c]pyridine-3-carbonyl)-amino]-acetic acid

¹H NMR (300 MHz, DMSO-d6): 12.96 (s, 1H), 9.58 (s, 1H), 8.70 (dd, 1H, J=4.2 Hz, 4.5 Hz), 8.26 (d, 1H, J=4.8 Hz), 7.77 (m, 1H), 4.99 (d, 2H, J=6 Hz). m/z=371 (M+H)

### [(1-Chloro-4-hydroxy-8-phenylmethanesulfonyl-benzo[4,5]thieno[3,2-c]pyridine-3-carbonyl)-amino]-acetic acid

¹H NMR (300 MHz, DMSO-d6) δ 13.00 (brs, 1H), 9.44 (s, 1H), 8.99 (s, 1H), 8.54 (d, J=8.4 Hz, 1H), 8.01 (d, J=8.4 Hz, 1H), 7.30-7.16 (m, 5H), 4.79 (s, 2H), 4.03 (d, J=6.3 Hz, 2H)

### [(1-Chloro-8-ethanesulfonyl-4-hydroxy-benzo[4,5]thieno[3,2-c]pyridine-3-carbonyl)-amino]-acetic acid

¹H NMR (300 MHz, DMSO-d6) δ 13.01 (brs, 1H), 9.58 (s, 1H), 9.31 (s, 1H), 8.59 (d, J=8.4 Hz, 1H), 8.18 (d, J=8.4 Hz, 1H), 4.03 (d, J=5.4 Hz, 2H), 3.42 (m, 2H), 1.16 (t, J=6.9 Hz, 3H)

### [(8-Benzenesulfonyl-1-chloro-4-hydroxy-benzo[4,5]thieno[3,2-c]pyridine-3-carbonyl)-amino]-acetic acid

¹H NMR (300 MHz, DMSO-d6) δ 9.36 (s, 1H), 8.50 (d, J=9 Hz, 1H), 8.19 (d, J=9 Hz, 1H), 8.03 (d, J=7.2 Hz, 2H), 7.70-7.61 (m, 3H), 4.01 (d, J=5.7 Hz, 2H)

### [(8-Benzenesulfinyl-1-chloro-4-hydroxy-benzo[4,5]thieno[3,2-c]pyridine-3-carbonyl)-amino]-acetic acid

¹H NMR (300 MHz, DMSO-d6) δ 9.13 (s, 1H), 8.32 (d, J=8.4 Hz, 1H), 7.86 (d; J=8.4 Hz, 1H), 7.78 (d, J=6.6 Hz, 2H), 7.57-7.50 (m, 3H), 3.99 (brs, 2H)

### Example 4

### Test and Administration

Biological Test

Biological activity of the compounds according to the present invention may be evaluated using any of the conventional known methods. The suitable assays have been widely known in the art. The following assays are described for the purpose of illustration, but are in no way intended to limit the scope of the present invention. The compounds of the present invention show activities in at least one of the following assays.

Cell-based Assay for HIFα Stabilization

Human cells induced from various tissues were inoculated into 35-mm culture dishes, respectively, and grown in standard culture media, for example, DMEM supplemented with 10% FBS under conditions of 37° C., 20% O₂ and 5% CO₂. Their cell layers grew into clusters, the media were replaced with OPTI-MEM media (Invitrogen Life Technologies, Carlsbad Calif.), and the cell layers were cultured at 37° C. for 24 hours under 20% O₂ and 5% CO₂ conditions. The compounds or 0. 013% DMSO were added to the existing media, and then cultured overnight.

After culturing, the media was removed, centrifuged, and stored for future analysis (see the following VEGF and EPO assays). The cells were washed twice with cold phosphate buffer saline (PBS), and then dissolved in a solution of 1 ml of 10 mM tris (pH 7.4), 1 mM EDTA, 150 mM NaCl, 0.5% IGEPAL (Sigma-Aldrich, St. Louis Mo.) and protease inhibitor mix (Roche Molecular Biochemicals) for 15 minutes while being kept in ice. Cell lysates were centrifuged at 4° C. for 5 minutes at a rotary speed of 3,000×g, and cytosol fractions (supernatant) were collected. Nuclei (pellets) were re-suspended and dissolved in a solution of 100 μl of 20 mM HEPES (pH 7.2), 400 mM NaCl, 1 mM EDTA, 1 mM dithiothreitol and protease mix (Roche Molecular Biochemicals), centrifuged at 4° C. for 5 minutes at a rotary speed of 13,000×g, and then nuclear protein fractions (supernatant) were collected.

Nucleus fractions were analyzed for HIF-1α using a QUANTIKINE immunoassay (R&D Systems, Inc., Minneapolis Minn.) according to the manufacturer's instructions.

HIF-PH2 (PHD2) Assay

Materials

HIF-PH2 (EGLN1) was expressed from E. coli cells, and purified using two process: an Ni-affinity chromatography column and a size-exclusion chromatography column.

HIF-PH2 (PHD2) Analysis (Fluorescence Polarization Method)

To evaluate activities of an HIF PH2 inhibitor, HIF PH2 enzyme that was first overexpressed by genetic recombination and then purified was used to perform an enzyme reaction. First, 200 nM HIF PH2 enzyme reacted with 50 nM peptide substrate (FITC-ACA-DLDLEALAPYIPADDDFQLR; SEQ ID NO.:1) in a reaction buffer (20 mM Tris-Cl (pH8.0), 100 mM NaCl, 0.5% Nonidet P40). At this time, 2 mM ascorbic acid and 5 mM ketoglutarate with 100 μM FeCl₂ or without FeCl₂, were used together with crude enzyme. A concentration of HIF PH2 inhibitor to be tested was treated and reacted at 30° C. for one hour. After the reaction, the resulting reaction product was boiled at 95° C. for one minute to suppress the enzyme reaction.

To determine whether prolyl hydroxylation occurs in the substrate as the secondary reaction, 500 nM GST-VBC (GST-VHL-Elongin B-Elongin C) protein was added to a reaction buffer (50 mM Tris-Cl (pH8.0), 120 mM NaCl, 0.5% Nonidet P40), and a GST-VBC binding reaction was carried out at room temperature for 30 minutes. After the reaction was completed, fluorescence polarization was determined at a wavelength of 485 nm/535 nm(ex/em) by using a Fusion-FP (Packard) system.

A fluorescence polarization value of a sample that is not treated with the HIF PH2 inhibitor was used as 100% control, and the activities of the HIF PH2 inhibitor were measured as percentage of the remaining HIF PH2 enzyme activity in samples treated with a concentration of the HIF PH2 inhibitor to be tested. The remaining HIF PH2 enzyme activities after the treatment with increasing concentrations of the HIF PH2 inhibitor was measured to calculate IC₅₀ of the HIF PH2 inhibitor, and then a concentration of the inhibitor was determined as IC₅₀, the concentration at which 50% of HIF PH2 enzyme activity is inhibited compared to the control. IC₅₀ data is given in Table 1.

TABLE 1
HIF PH2 inhibition activity

| Compound | IC₅₀ |
|---|---|
| [(4-Hydroxy-benzo[4,5]furo[3,2-c]pyridine-3-carbonyl)-amino]-acetic acid | B |
| [(4-Hydroxy-benzo[4,5]thieno[3,2-c]pyridine-3-carbonyl)-amino]-acetic acid | A |
| [(1-Chloro-4-hydroxy-benzo[4,5]thieno[3,2-c]pyridine-3-carbonyl)-amino]-acetic acid | A |
| [(7-Hydroxy-furo[3,2-c]pyridine-6-carbonyl)-amino]-acetic acid | D |
| [(7-Hydroxy-3-phenyl-furo[3,2-c]pyridine-6-carbonyl)-amino]-acetic acid | C |
| [(4-Hydroxy-benzo[4,5]furo[3,2-c]pyridine-3-carbonyl)-amino]-acetic acid | B |
| [(4-Hydroxy-benzo[4,5]thieno[3,2-c]pyridine-3-carbonyl)-amino]-acetic acid | A |
| [(1-Chloro-4-hydroxy-benzo[4,5]thieno[3,2-c]pyridine-3-carbonyl)-amino]-acetic acid | A |
| [(7-Hydroxy-furo[3,2-c]pyridine-6-carbonyl)-amino]-acetic acid | D |
| (S)-2-[(7-Hydroxy-furo[3,2-c]pyridine-6-carbonyl)-amino]-propionic acid | D |
| [(4-Hydroxy-1-phenyl-1H-pyrazolo[3,4-c]pyridine-5-carbonyl)-amino]-acetic acid | A |
| [(7-Chloro-4-hydroxy-1-phenyl-1H-pyrazolo[3,4-c]pyridine-5-carbonyl)-amino]-acetic acid | A |
| [(1-Chloro-4-hydroxy-8-nitro-benzo[4,5]thieno[3,2-c]pyridine-3-carbonyl)-amino]-acetic acid | A |
| 3-(Carboxymethyl-carbamoyl)-1-chloro-4-hydroxy-benzo[4,5]thieno[3,2-c]pyridin-8-yl- | A |
| ammonium | |
| [(1-Bromo-4-hydroxy-benzo[4,5]thieno[3,2-c]pyridine-3-carbonyl)-amino]-acetic acid | A |
| (S)-2-[(1-Chloro-4-hydroxy-benzo[4,5]thieno[3,2-c]pyridine-3-carbonyl)-amino]-propionic acid | B |
| (S)-2-[(1-Bromo-4-hydroxy-benzo[4,5]thieno[3,2-c]pyridine-3-carbonyl)-amino]-propionic acid | B |
| [(1-Chloro-8-fluoro-4-hydroxy-benzo[4,5]thieno[3,2-c]pyridine-3-carbonyl)-amino]-acetic acid | A |
| [(1-Cyano-4-hydroxy-benzo[4,5]thieno[3,2-c]pyridine-3-carbonyl)-amino]-acetic acid | A |
| [(1-Benzenesulfonyl-7-chloro-4-hydroxy-1H-pyrrolo[2,3-c]pyridine-5-carbonyl)-amino]-acetic | A |
| acid | |
| [(1-Benzenesulfonyl-7-chloro-4-hydroxy-1H-pyrrolo[2,3-c]pyridine-5-carbonyl)-amino]-acetic | A |
| acid methyl ester | |
| [(7-Chloro-4-hydroxy-1H-pyrrolo[2,3-c]pyridine-5-carbonyl)-amino]-acetic acid | D |
| [(4-Amino-1-bromo-benzo[4,5]thieno[3,2-c]pyridine-3-carbonyl)-amino]-acetic acid | D |
| 1-Bromo-4-hydroxy-benzo[4,5]thieno[3,2-c]pyridine-3-carboxylic acid (pyridin-3-ylmethyl)- | B |
| amide | |
| [(1-Bromo-4-fluoro-benzo[4,5]thieno[3,2-c]pyridine-3-carbonyl)-amino]-acetic acid | A |
| 1-Bromo-4-hydroxy-benzo[4,5]thieno[3,2-c]pyridine-3-carboxylic acid [2-(4-methoxy-benzyl)- | A |
| 2H-tetrazol-5-ylmethyl]-amide | |
| 1-Bromo-4-hydroxy-benzo[4,5]thieno[3,2-c]pyridine-3-carboxylic acid [1-(4-methoxy-benzyl)- | B |
| 1H-tetrazol-5-ylmethyl]-amide | |
| 1-Bromo-4-hydroxy-benzo[4,5]thieno[3,2-c]pyridine-3-carboxylic acid (pyridin-2-ylmethyl)- | A |
| amide | |
| 1-Bromo-4-hydroxy-benzo[4,5]thieno[3,2-c]pyridine-3-carboxylic acid benzylamide | B |
| [(1-Chloro-8-dimethylamino-4-hydroxy-benzo[4,5]thieno[3,2-c]pyridine-3-carbonyl)-amino]- | A |
| acetic acid | |
| [(1-Chloro-8-diethylamino-4-hydroxy-benzo[4,5]thieno[3,2-c]pyridine-3-carbonyl)-amino]- | A |
| acetic acid | |
| [(8-Acetylamino-1-chloro-4-hydroxy-benzo[4,5]thieno[3,2-c]pyridine-3-carbonyl)-amino]- | A |
| acetic acid | |
| [(4-Chloro-benzo[4,5]thieno[3,2-c]pyridine-3-carbonyl)-hydroxy-amino]-acetic acid | D |
| [(1-Chloro-6-fluoro-4-hydroxy-benzo[4,5]thieno[3,2-c]pyridine-3-carbonyl)-amino]-acetic acid | A |
| [(1-Chloro-7-fluoro-4-hydroxy-benzo[4,5]thieno[3,2-c]pyridine-3-carbonyl)-amino]-acetic acid | A |
| [(1-Chloro-9-fluoro-4-hydroxy-benzo[4,5]thieno[3,2-c]pyridine-3-carbonyl)-amino]-acetic acid | A |
| [(4-Hydroxy-1-pyridin-2-yl-1H-pyrazolo[3,4-c]pyridine-5-carbonyl)-amino]-acetic acid | A |
| [(4-Hydroxy-1-methyl-benzo[4,5]thieno[3,2-c]pyridine-3-carbonyl)-amino]-acetic acid | A |
| [Hydroxy-(4-hydroxy-benzo[4,5]thieno[3,2-c]pyridine-3-carbonyl)-amino]-acetic acid | C |
| [(1-Chloro-4,8-dihydroxy-benzo[4,5]thieno[3,2-c]pyridine-3-carbonyl)-amino]-acetic acid | A |
| [(1-Chloro-4-hydroxy-7-methoxy-benzo[4,5]thieno[3,2-c]pyridine-3-carbonyl)-amino]-acetic | A |
| acid | |
| [(1-Chloro-8-hydroxy-4-methoxy-benzo[4,5]thieno[3,2-c]pyridine-3-carbonyl)-amino]-acetic | C |
| acid | |
| [(1-Chloro-8-hydroxy-4-isopropoxy-benzo[4,5]thieno[3,2-c]pyridine-3-carbonyl)-amino]-acetic | C |
| acid | |
| [(1-Chloro-4,7-dihydroxy-benzo[4,5]thieno[3,2-c]pyridine-3-carbonyl)-amino]-acetic acid | A |
| [(1-Chloro-4-hydroxy-7-isopropoxy-benzo[4,5]thieno[3,2-c]pyridine-3-carbonyl)-amino]-acetic | A |
| acid | |
| [(7-Fluoro-4-hydroxy-1-methyl-benzo[4,5]thieno[3,2-c]pyridine-3-carbonyl)-amino]-acetic acid | A |
| [(1-Chloro-8-ethylamino-4-hydroxy-benzo[4,5]thieno[3,2-c]pyridine-3-carbonyl)-amino]-acetic | A |
| acid | |
| [(8-Benzenesulfonylamino-1-chloro-4-hydroxy-benzo[4,5]thieno[3,2-c]pyridine-3-carbonyl)- | A |
| amino]-acetic acid | |
| [(8-Benzylamino-1-chloro-4-hydroxy-benzo[4,5]thieno[3,2-c]pyridine-3-carbonyl)-amino]- | A |
| acetic acid | |
| [(1-Chloro-4-hydroxy-8-trifluoromethyl-benzo[4,5]thieno[3,2-c]pyridine-3-carbonyl)-amino]- | A |
| acetic acid | |
| [(1-Chloro-4-hydroxy-8-phenylmethanesulfonyl-benzo[4,5]thieno[3,2-c]pyridine-3-carbonyl)- | B |
| amino]-acetic acid | |
| [(1-Chloro-8-ethanesulfonyl-4-hydroxy-benzo[4,5]thieno[3,2-c]pyridine-3-carbonyl)-amino]- | B |
| acetic acid | |
| [(8-Benzenesulfonyl-1-chloro-4-hydroxy-benzo[4,5]thieno[3,2-c]pyridine-3-carbonyl)-amino]- | A |
| acetic acid | |
| [(8-Benzenesulfinyl-1-chloro-4-hydroxy-benzo[4,5]thieno[3,2-c]pyridine-3-carbonyl)-amino]- | A |
| acetic acid | |

A = 0-25 μM;
B = 26-100 μM;
C = 101-200 μM;
D = >201 μM

### Human EPO Immunoassay

Human cells derived from hepatocarcinoma (Hep3B) tissue (see, e.g., American Type Culture Collection, Manassas Va.) were grown at 37° C., 20% O₂, 5% CO2 in DMEM (GIBCO)+10% FBS, 4.5 g/L D-Glucose; L-Glutamate and 110 mg/L sodium pyruvate. Ninety-six well plates were seeded with 4×10⁴ HEP3B cells/well. The media was removed and replaced with DMEM+10% FBS. Compounds were added to wells at concentrations between 1 μM-100 μM for a 24-hour incubation. Cell culture media was harvested and EPO concentration was determined using a Human Erythropoietin Quantikine IVD ELISA Kit (R&D Systems®, Minneapolis, Minn.) following the manufacturer's instructions for the benchtop assay. Results, including both the raw data and in comparison to control (at 100 μM), are shown in Table 2.

TABLE 2
EPO Induction

| | Fold Induction | |
|---|---|---|
| Compound at 100 μM | over control | EPO (mIU/ml) |
| [(4-Hydroxy-benzo[4,5]furo[3,2-c]pyridine-3-carbonyl)- | E | I |
| amino]-acetic acid | | |
| [(4-Hydroxy-benzo[4,5]thieno[3,2-c]pyridine-3-carbonyl)- | E | I |
| amino]-acetic acid | | |
| [(1-Chloro-4-hydroxy-benzo[4,5]thieno[3,2-c]pyridine-3- | F | J |
| carbonyl)-amino]-acetic acid | | |
| [(1-Chloro-8-fluoro-4-hydroxy-benzo[4,5]thieno[3,2- | F | J |
| c]pyridine-3-carbonyl)-amino]-acetic acid | | |
| [(1-Benzenesulfonyl-7-chloro-4-hydroxy-1H-pyrrolo[2,3- | E | I |
| c]pyridine-5-carbonyl)-amino]-acetic acid | | |
| [(1-Chloro-8-diethylamino-4-hydroxy-benzo[4,5]thieno[3,2- | F | J |
| c]pyridine-3-carbonyl)-amino]-acetic acid | | |
| [(1-Chloro-6-fluoro-4-hydroxy-benzo[4,5]thieno[3,2- | E | I |
| c]pyridine-3-carbonyl)-amino]-acetic acid | | |
| [(1-Chloro-7-fluoro-4-hydroxy-benzo[4,5]thieno[3,2- | G | K |
| c]pyridine-3-carbonyl)-amino]-acetic acid | | |
| [(1-Chloro-9-fluoro-4-hydroxy-benzo[4,5]thieno[3,2- | H | K |
| c]pyridine-3-carbonyl)-amino]-acetic acid | | |
| [(4-Hydroxy-1-pyridin-2-yl-1H-pyrazolo[3,4-c]pyridine-5- | E | I |
| carbonyl)-amino]-acetic acid | | |
| [(4-Hydroxy-1-methyl-benzo[4,5]thieno[3,2-c]pyridine-3- | H | J |
| carbonyl)-amino]-acetic acid | | |
| [(1-Chloro-4,8-dihydroxy-benzo[4,5]thieno[3,2-c]pyridine-3- | E | I |
| carbonyl)-amino]-acetic acid | | |
| [(1-Chloro-4-hydroxy-7-methoxy-benzo[4,5]thieno[3,2- | F | I |
| c]pyridine-3-carbonyl)-amino]-acetic acid | | |
| [(1-Chloro-4,7-dihydroxy-benzo[4,5]thieno[3,2-c]pyridine-3- | E | I |
| carbonyl)-amino]-acetic acid | | |
| [(1-Chloro-4-hydroxy-7-isopropoxy-benzo[4,5]thieno[3,2- | F | L |
| c]pyridine-3-carbonyl)-amino]-acetic acid | | |
| [(7-Fluoro-4-hydroxy-1-methyl-benzo[4,5]thieno[3,2- | F | L |
| c]pyridine-3-carbonyl)-amino]-acetic acid | | |
| [(1-Chloro-8-ethylamino-4-hydroxy-benzo[4,5]thieno[3,2- | G | K |
| c]pyridine-3-carbonyl)-amino]-acetic acid | | |
| [(8-Benzenesulfonylamino-1-chloro-4-hydroxy- | E | I |
| benzo[4,5]thieno[3,2-c]pyridine-3-carbonyl)-amino]-acetic | | |
| acid | | |
| [(1-Chloro-4-hydroxy-8-trifluoromethyl-benzo[4,5]thieno[3,2- | F | J |
| c]pyridine-3-carbonyl)-amino]-acetic acid | | |

E = 0-20;
F = 21-50;
G = 51-70;
H = >71
I = 0-20 mIU/ml;
J = 21-40 mIU/ml;
K = 41-60 mIU/ml;
L = >61 mIU/ml

Cell-Based Assay for VEGF and EPO Reporters

A luciferase assay was used to determine the changes in transcription amount of EPO and VEGF genes in cells. For the luciferase assay, a human HIF I a gene was first cloned into an animal cell expression vector, pFlag-CMV, to prepare a pFlag-HIF1α vector, and a hypoxia responsible element (HRE) sequence of an EPO gene 3′-enhancer domain was then cloned upstream of luciferase and TK promoter genes to prepare a pEPO HRE-Luc expression vector. Also, the promoter and luciferase domains of the VEGF gene were cloned into a pGL3-basic vector to prepare a pVEGF-Luc expression vector. HeLa cells were seed-cultured in a medium dish to grow to about 70-80% density one day before the HeLa cells were to be used. The HeLa cells were transfected with each of the prepared pEPO HRE-Luc and pVEGF-Luc expression vectors together with the pFlag-HIF1α and the Renilla luciferase expression vector (Promega, Madison, Wis., USA), by using LipofectAMINE PLUS™ (Invitrogen Life Technologies, Carlsbad Calif.). Three hours after the transfection, the medium was exchanged with DMEM, 1% Penicillin-Streptomycin in 10% FBS supplemented with serum. At this time, cultured cells were treated with each of the compounds at the concentrations indicated. Then, the cells were cultured for 24 hrs in an incubator that was maintained under conditions of 37° C., 20% O₂ and 5% CO₂. After culturing, cells were washed twice with cold phosphate buffer saline (PBS). Luciferase activity of the cells was measured using the dual luciferase assay system (Promega, Madison, Wis., USA). The results of this analysis are shown in Table 3.

TABLE 3
VEGF Induction

| | VEGF 1 | VEGF 2 | EPO |
|---|---|---|---|
| | fold induction | fold induction | fold induction |
| Compound 100 μM | over control | over control | over control |
| [(1-Chloro-4-hydroxy-benzo[4,5]thieno[3,2- | O | O | O |
| c]pyridine-3-carbonyl)-amino]-acetic acid | | | |
| [(4-Hydroxy-1-phenyl-1H-pyrazolo[3,4- | N | | O |
| c]pyridine-5-carbonyl)-amino]-acetic acid | | | |
| [(7-Chloro-4-hydroxy-1-phenyl-1H- | N | | O |
| pyrazolo[3,4-c]pyridine-5-carbonyl)-amino]- | | | |
| acetic acid | | | |
| [(1-Chloro-4-hydroxy-8-nitro- | O | | N |
| benzo[4,5]thieno[3,2-c]pyridine-3-carbonyl)- | | | |
| amino]-acetic acid | | | |
| 3-(Carboxymethyl-carbamoyl)-1-chloro-4- | N | | N |
| hydroxy-benzo[4,5]thieno[3,2-c]pyridin-8-yl- | | | |
| ammonium | | | |
| [(1-Bromo-4-hydroxy-benzo[4,5]thieno[3,2- | 4 | | O |
| c]pyridine-3-carbonyl)amino]-acetic acid | | | |
| (S)-2-[(1-Chloro-4-hydroxy- | O | | N |
| benzo[4,5]thieno[3,2-c]pyridine-3-carbonyl)- | | | |
| amino]-propionic acid | | | |
| (S)-2-[(1-Bromo-4-hydroxy- | O | | N |
| benzo[4,5]thieno[3,2-c]pyridine-3-carbonyl)- | | | |
| amino]-propionic acid | | | |
| [(1-Chloro-8-fluoro-4-hydroxy- | N | | O |
| benzo[4,5]thieno[3,2-c]pyridine-3-carbonyl)- | | | |
| amino]-acetic acid | | | |
| [(1-Cyano-4-hydroxy-benzo[4,5]thieno[3,2- | N | | M |
| c]pyridine-3-carbonyl)-amino]-acetic acid | | | |
| 1-Bromo-4-hydroxy-benzo[4,5]thieno[3,2- | N | | M |
| c]pyridine-3-carboxylic acid [2-(4-methoxy- | | | |
| benzyl)-2H-tetrazol-5-ylmethyl]-amide | | | |
| 1-Bromo-4-hydroxy-benzo[4,5]thieno[3,2- | M | | M |
| c]pyridine-3-carboxylic acid [1-(4-methoxy- | | | |
| benzyl)-1H-tetrazol-5-ylmethyl]-amide | | | |
| 1-Bromo-4-hydroxy-benzo[4,5]thieno[3,2- | N | | M |
| c]pyridine-3-carboxylic acid (pyridin-2- | | | |
| ylmethyl)-amide | | | |
| 1-Bromo-4-hydroxy-benzo[4,5]thieno[3,2- | M | | M |
| c]pyridine-3-carboxylic acid benzylamide | | | |
| [(1-Chloro-8-dimethylamino-4-hydroxy- | O | | O |
| benzo[4,5]thieno[3,2-c]pyridine-3-carbonyl)- | | | |
| amino]-acetic acid | | | |
| [(8-Acetylamino-1-chloro-4-hydroxy- | | M | |
| benzo[4,5]thieno[3,2-c]pyridine-3-carbonyl)- | | | |
| amino]-acetic acid | | | |
| [(1-Chloro-6-fluoro-4-hydroxy- | N | | N |
| benzo[4,5]thieno[3,2-c]pyridine-3-carbonyl)- | | | |
| amino]-acetic acid | | | |
| [(1-Chloro-7-fluoro-4-hydroxy- | O | | O |
| benzo[4,5]thieno[3,2-c]pyridine-3-carbonyl)- | | | |
| amino]-acetic acid | | | |
| [(1-Chloro-9-fluoro-4-hydroxy- | | N | |
| benzo[4,5]thieno[3,2-c]pyridine-3-carbonyl)- | | | |
| amino]-acetic acid | | | |

M = 0-2.0;
N = 2.1-4.0;
O = >4.1

What is claimed is:

1. A compound of Formula I: 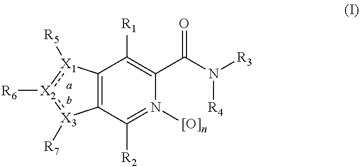
or a pharmaceutically acceptable salt, ester, amide, or prodrug thereof,
wherein
n is 0 or 1;
R₁ is —OR₈ or halo;
R₂ is selected from the group consisting of hydrogen, optionally substituted alkyl, optionally substituted cycloalkyl, optionally substituted aryl, halo, and cyano;
R₃ is selected from the group consisting of optionally substituted cycloalkyl, optionally substituted aryl, optionally substituted heteroaryl, —CR₉R₁₀R₁₁, and —CR₉R₁₀—C(═O)OR₁₂;
R₄ is hydrogen or —OR₈;
R₈ is selected from the group consisting of hydrogen, optionally substituted alkyl, optionally substituted cycloalkyl, and optionally substituted aryl;
R₉ and R₁₀ are each independently selected from the group consisting of hydrogen, optionally substituted alkyl, optionally substituted cycloalkyl, and optionally substituted aryl;
R₁₁ is selected from the group consisting of optionally substituted aryl, and optionally substituted heteroaryl;
R₁₂ is selected from the group consisting of hydrogen, optionally substituted alkyl, optionally substituted cycloalkyl, optionally substituted aryl, and optionally substituted heteroaryl; and
i) X₁ is sulfur;
R₅ does not exist;
X₂ and X₃ are both carbon;
R₆ and R₇ taken together along with the carbon atoms to which they are attached form a ring of formula 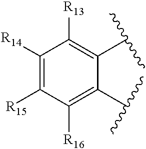
R₁₃ and R₁₄ are each independently selected from the group consisting of hydrogen, optionally substituted alkyl, optionally substituted cycloalkyl, optionally substituted aryl, halo, —OR₈, and cyano;
R₁₅ and R₁₆ are each independently selected from the group consisting of hydrogen, optionally substituted alkyl, optionally substituted cycloalkyl, optionally substituted aryl, halo, perhaloalkyl, —OR₈, —NO₂, —N(R₈)₂, —NHC(═O)R₈, —NH(SO₂)Ar, and —(CR₉R₁₀)ₘ—S(═O)—(CR₉R₁₀)ₚ—R₈,
—(CR₉R₁₀)ₘ—S(═O)₂—(CR₉R₁₀)ₚ—R₈, cyano, wherein Ar is an optionally substituted aryl, and m and p is each independently 0-10, inclusive; and
bond a is a single bond and bond b is a double bond; or
ii) X₁ is oxygen;
R₅ does not exist;
X₂ and X₃ are both carbon;
R₆ is selected from the group consisting of hydrogen, optionally substituted alkyl, optionally substituted cycloalkyl, optionally substituted aryl, optionally substituted heteroaryl, and —OR₈;
R₇ is selected from the group consisting of hydrogen, optionally substituted alkyl, optionally substituted cycloalkyl, optionally substituted aryl, optionally substituted heteroaryl, and —SO₂—Ar, wherein Ar is an optionally substituted aryl; or
R₆ and R₇ taken together along with the carbon atoms to which they are attached form an optionally substituted phenyl; and
bond a is a single bond and bond b is a double bond; or
iii) X₁ is carbon and X₂ and X₃ are both nitrogen;
R₅ is selected from the group consisting of hydrogen, optionally substituted alkyl, optionally substituted cycloalkyl, optionally substituted aryl, and optionally substituted heteroaryl;
R₆ does not exist;
R₇ is selected from the group consisting of hydrogen, optionally substituted alkyl, optionally substituted cycloalkyl, optionally substituted aryl, optionally substituted heteroaryl, and —SO₂—Ar, wherein Ar is an optionally substituted aryl; and
bond a is a single bond and bond b is a double bond.

2. The compound of claim 1, wherein R₁ is selected from the group consisting of fluoro, chloro, bromo, and iodo.

3. The compound of claim 1, wherein R₁ is —OR₈ and R₈ is selected from the group consisting of hydrogen, and optionally substituted alkyl.

4. The compound of claim 1, wherein R₂ is selected from the group consisting of hydrogen, optionally substituted alkyl, fluoro, chloro, bromo, iodo, and cyano.

5. The compound of claim 1, wherein R₂ is selected from the group consisting of hydrogen, methyl, chloro, bromo, and cyano.

6. The compound of claim 1, wherein R₃ is selected from the group consisting of optionally substituted aryl, optionally substituted heteroaryl, —CR₉R₁₀R₁₁, and —CR₉R₁₀—C(═O)OR₁₂.

7. The compound of claim 6, wherein R₃ is optionally substituted phenyl or optionally substituted pyridyl.

8. The compound of claim 6, wherein R₉ and R₁₀ is each independently selected from the group consisting of hydrogen, optionally substituted alkyl, and optionally substituted aryl.

9. The compound of claim 6, wherein R₉ is hydrogen and R₁₀ is hydrogen or methyl.

10. The compound of claim 6, wherein R₁₁ is selected from the group consisting of optionally substituted phenyl, optionally substituted pyridyl, and optionally substituted tetrazolyl.

11. The compound of claim 6, wherein R₁₁ is selected from the group consisting of phenyl, pyridyl, 1H-tetrazol-5-yl, and [2-(4-methoxy-benzyl)-2H-tetrazol-5-yl.

12. The compound of claim 6, wherein R₁₂ is hydrogen or optionally substituted alkyl.

13. The compound of claim 6, wherein R₁₂ is hydrogen or methyl.

14. The compound of claim 1, wherein R₄ is —OR₈ and R₈ is selected from the group consisting of hydrogen, and optionally substituted alkyl.

15. The compound of claim 14, wherein R₄ is hydrogen or hydroxyl.

16. The compound of claim 1, wherein R₇ is selected from the group consisting of hydrogen, phenyl, pyridyl, and —SO₂—C₆H₅.

17. The compound of claim 1, wherein 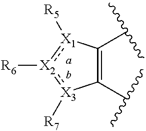
is selected from the group consisting of

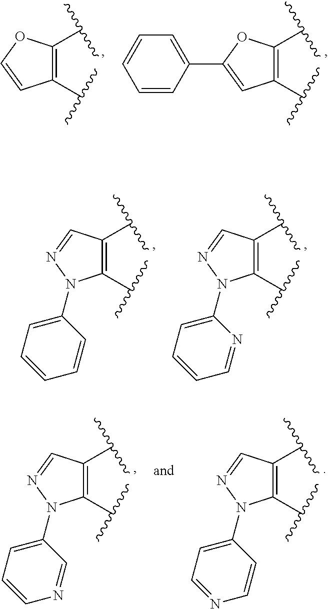

18. The compound of claim 1, wherein R₁₃ and R₁₄ is each independently selected from the group consisting of hydrogen, halogen, optionally substituted alkyl, and optionally substituted aryl.

19. The compound of claim 1, wherein R₁₃ and R₁₄ are each independently selected from the group consisting of hydrogen, fluoro, chloro, bromo, and iodo.

20. The compound of claim 1, wherein R₁₅ is selected from the group consisting of hydrogen, halo, perhaloalkyl, —OR₈, —NO₂, —N(R₈)₂, —NHC(═O)R₈, —NH(SO₂)Ar, —(CR₉R₁₀)ₘ—S(═O)—(CR₉R₁₀)ₚ—R₈, and —(CR₉R₁₀)ₘ—S(═O)₂—(CR₉R₁₀)ₚ—R₈.

21. The compound of claim 1, wherein R₁₅ is selected from the group consisting of hydrogen, fluororo, trifluoromethyl, —OH, —NH₂, —NH(CH₂CH₃), —NH(CH₂—C₆H₅), —N(CH₃)₂, —N(CH₂CH₃)₂, —NH(SO₂)—C₆H₅, —NHC(═O)CH₃, and —S(═O)-Ph, —S(═O)₂—CH₂CH₃, —S(═O)₂-Ph, and —S(═O)₂—CH₂Ph.

22. The compound of claim 1, wherein R₁₆ is selected from the group consisting of hydrogen, optionally substituted alkyl, optionally substituted aryl, and —(CR₉R₁₀)ₘ—S(═O)₂—(CR₉R₁₀)ₚ—R₈.

23. The compound of claim 1, wherein R₁₆ is hydrogen or —S(═O)₂—CH₂CH₃.

24. The compound of claim 1, wherein the ring of formula 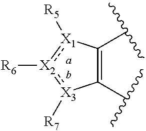
is selected from the group consisting of

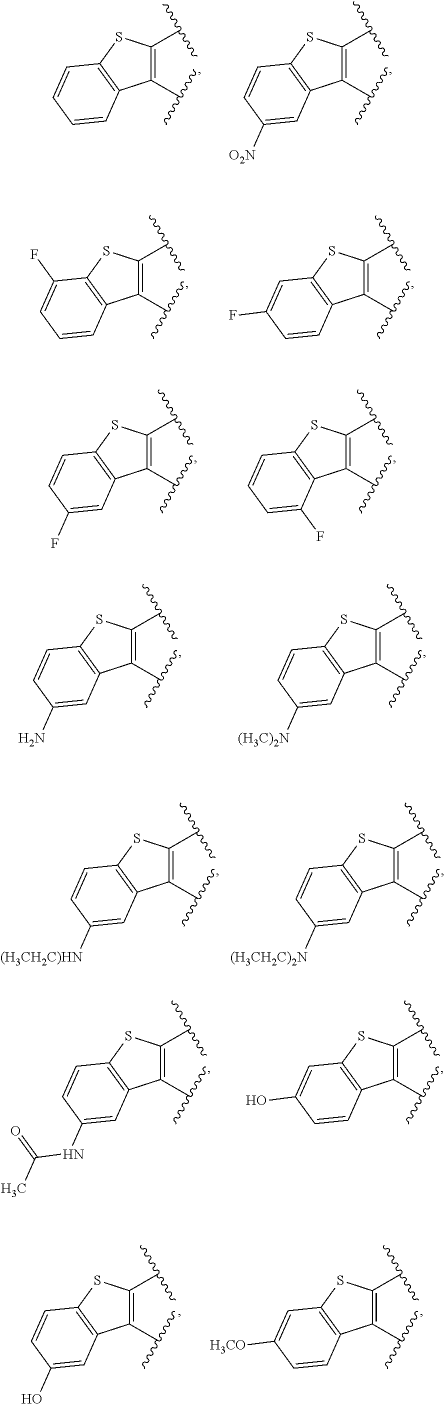

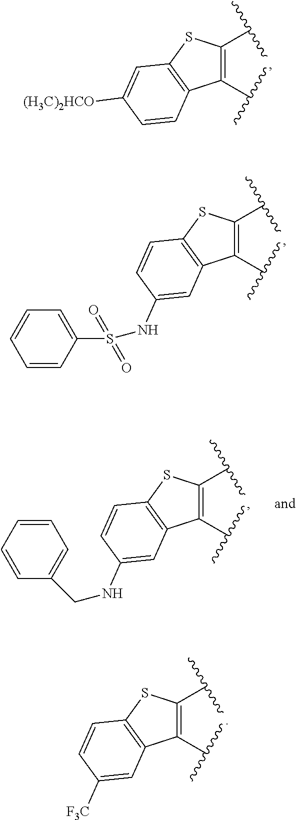

25. The compound of claim 1, wherein n is 0.

26. A compound selected from the group consisting of
[(4-Hydroxy-benzo[4,5]furo[3,2-c]pyridine-3-carbonyl)-amino]-acetic acid,
[(4-Hydroxy-benzo[4,5]thieno[3,2-c]pyridine-3-carbonyl)-amino]-acetic acid,
[(1-Chloro-4-hydroxy-benzo[4,5]thieno[3,2-c]pyridine-3-carbonyl)-amino]-acetic acid,
[(7-Hydroxy-furo[3,2-c]pyridine-6-carbonyl)-amino]-acetic acid,
[(7-Hydroxy-2-phenyl-furo[3,2-c]pyridine-6-carbonyl)-amino]-acetic acid,
(S)-2-[(7-Hydroxy-furo[3,2-c]pyridine-6-carbonyl)-amino]-propionic acid,
[(4-Hydroxy-1-phenyl-1H-pyrazolo[3,4-c]pyridine-5-carbonyl)-amino]-acetic acid,
[(7-Chloro-4-hydroxy-1-phenyl-1H-pyrazolo[3,4-c]pyridine-5-carbonyl)-amino]-acetic acid,
[(1-Chloro-4-hydroxy-8-nitro-benzo[4,5]thieno[3,2-c]pyridine-3-carbonyl)-amino]-acetic acid,
3-(Carboxymethyl-carbamoyl)-1-chloro-4-hydroxy-benzo[4,5]thieno[3,2-c]pyridin-8-yl-ammonium,
[(1-Bromo-4-hydroxy-benzo[4,5]thieno[3,2-c]pyridine-3-carbonyl)-amino]-acetic acid,
(S)-2-[(1-Chloro-4-hydroxy-benzo[4,5]thieno[3,2-c]pyridine-3-carbonyl)-amino]-propionic acid,
(S)-2-[(1-Bromo-4-hydroxy-benzo[4,5]thieno[3,2-c]pyridine-3-carbonyl)-amino]-propionic acid,
[(1-Chloro-8-fluoro-4-hydroxy-benzo[4,5]thieno[3,2-c]pyridine-3-carbonyl)-amino]-acetic acid,
[(1-Cyano-4-hydroxy-benzo[4,5]thieno[3,2-c]pyridine-3-carbonyl)-amino]-acetic acid,
[(4-Amino-1-bromo-benzo[4,5]thieno[3,2-c]pyridine-3-carbonyl)-amino]-acetic acid,
1-Bromo-4-hydroxy-benzo[4,5]thieno[3,2-c]pyridine-3-carboxylic acid (pyridin-3-ylmethyl)-amide,
[(1-Bromo-4-fluoro-benzo[4,5]thieno[3,2-c]pyridine-3-carbonyl)-amino]-acetic acid,
1-Bromo-4-hydroxy-benzo[4,5]thieno[3,2-c]pyridine-3-carboxylic acid [2-(4-methoxy-benzyl)-2H-tetrazol-5-ylmethyl]-amide,
1-Bromo-4-hydroxy-benzo[4,5]thieno[3,2-c]pyridine-3-carboxylic acid [1-(4-methoxy-benzyl)-1H-tetrazol-5-ylmethyl]-amide,
1-Bromo-4-hydroxy-benzo[4,5]thieno[3,2-c]pyridine-3-carboxylic acid (pyridin-2-ylmethyl)-amide,
1-Bromo-4-hydroxy-benzo[4,5]thieno[3,2-c]pyridine-3-carboxylic acid (1H-tetrazol-5-ylmethyl)-amide,
1-Bromo-4-hydroxy-benzo[4,5]thieno[3,2-c]pyridine-3-carboxylic acid pyridin-2-ylamide,
1-Bromo-4-hydroxy-benzo[4,5]thieno[3,2-c]pyridine-3-carboxylic acid pyridin-3-ylamide,
1-Bromo-4-hydroxy-benzo[4,5]thieno[3,2-c]pyridine-3-carboxylic acid phenylamide,
1-Bromo-4-hydroxy-benzo[4,5]thieno[3,2-c]pyridine-3-carboxylic acid benzylamide,
[(1-Chloro-8-dimethylamino-4-hydroxy-benzo[4,5]thieno[3,2-c]pyridine-3-carbonyl)-amino]-acetic acid,
[(1-Chloro-8-diethylamino-4-hydroxy-benzo[4,5]thieno[3,2-c]pyridine-3-carbonyl)-amino]-acetic acid,
[(8-Acetylamino-1-chloro-4-hydroxy-benzo[4,5]thieno[3,2-c]pyridine-3-carbonyl)-amino]-acetic acid,
[(4-Chloro-benzo[4,5]thieno[3,2-c]pyridine-3-carbonyl)-hydroxy-amino]-acetic acid,
[(1-Chloro-6-fluoro-4-hydroxy-benzo[4,5]thieno[3,2-c]pyridine-3-carbonyl)-amino]-acetic acid,
[(1-Chloro-7-fluoro-4-hydroxy-benzo[4,5]thieno[3,2-c]pyridine-3-carbonyl)-amino]-acetic acid,
[(1-Chloro-9-fluoro-4-hydroxy-benzo[4,5]thieno[3,2-c]pyridine-3-carbonyl)-amino]-acetic acid,
[(4-Hydroxy-1-pyridin-2-yl-1H-pyrazolo[3,4-c]pyridine-5-carbonyl)-amino]-acetic acid,
[(4-Hydroxy-1-methyl-benzo[4,5]thieno[3,2-c]pyridine-3-carbonyl)-amino]-acetic acid,
[Hydroxy-(4-hydroxy-benzo[4,5]thieno[3,2-c]pyridine-3-carbonyl)-amino]-acetic acid,
[(1-Chloro-4,8-dihydroxy-benzo[4,5]thieno[3,2-c]pyridine-3-carbonyl)-amino]-acetic acid,
[(1-Chloro-4-hydroxy-7-methoxy-benzo[4,5]thieno[3,2-c]pyridine-3-carbonyl)-amino]-acetic acid,
[(1-Chloro-8-hydroxy-4-methoxy-benzo[4,5]thieno[3,2-c]pyridine-3-carbonyl)-amino]-acetic acid,
[(1-Chloro-8-hydroxy-4-isopropoxy-benzo[4,5]thieno[3,2-c]pyridine-3-carbonyl)-amino]-acetic acid,
[(1-Chloro-4,7-dihydroxy-benzo[4,5]thieno[3,2-c]pyridine-3-carbonyl)-amino]-acetic acid,
[(1-Chloro-4-hydroxy-7-isopropoxy-benzo[4,5]thieno[3,2-c]pyridine-3-carbonyl)-amino]-acetic acid,
[(7-Fluoro-4-hydroxy-1-methyl-benzo[4,5]thieno[3,2-c]pyridine-3-carbonyl)-amino]-acetic acid,
[(1-Chloro-8-ethylamino-4-hydroxy-benzo[4,5]thieno[3,2-c]pyridine-3-carbonyl)-amino]-acetic acid,
[(8-Benzenesulfonylamino-1-chloro-4-hydroxy-benzo[4,5]thieno[3,2-c]pyridine-3-carbonyl)-amino]-acetic acid,
[(8-Benzylamino-1-chloro-4-hydroxy-benzo[4,5]thieno[3,2-c]pyridine-3-carbonyl)-amino]-acetic acid,
[(1-Chloro-4-hydroxy-8-trifluoromethyl-benzo[4,5]thieno[3,2-c]pyridine-3-carbonyl)-amino]-acetic acid,
[(1-Chloro-7-fluoro-4-hydroxy-2-oxy-benzo[4,5]thieno[3,2-c]pyridine-3-carbonyl)-amino]-acetic acid,
[(1-Chloro-4-hydroxy-8-phenylmethanesulfonyl-benzo[4,5]thieno[3,2-c]pyridine-3-carbonyl)-amino]-acetic acid,
[(1-Chloro-8-ethanesulfonyl-4-hydroxy-benzo[4,5]thieno[3,2-e]pyridine-3-carbonyl)-amino]-acetic acid,
[(8-Benzenesulfonyl-1-chloro-4-hydroxy-benzo[4,5]thieno[3,2-c]pyridine-3-carbonyl)-amino]-acetic acid,
[(8-Benzenesulfinyl-1-chloro-4-hydroxy-benzo[4,5]thieno[3,2-c]pyridine-3-carbonyl)-amino]-acetic acid, and
or a pharmaceutically acceptable salt, ester, amide, or prodrug thereof.

27. A pharmaceutical composition comprising a therapeutically effective amount of at least one compound of Formula I-V, or a pharmaceutically acceptable salt, ester, amide, or prodrug thereof, and a physiologically acceptable carrier, diluent, or excipient.

28. A method of modulating a level of HIF in a subject comprising identifying a subject in need thereof and administering to the subject, or contacting the subject with, an amount of at least one compound of Formula I-V, or a pharmaceutically acceptable salt, ester, amide, or prodrug thereof, sufficient to modulate the level of HIF in the subject.

29. A method of modulating an amount of HIF in a cell comprising administering to the cell, or contacting the cell with, an amount of at least one compound of Formula I-V, or a pharmaceutically acceptable salt, ester, amide, or prodrug thereof, sufficient to modulate the amount of HIF in the cell.

30. The method of claim 29, wherein the amount of HIF in the cell is increased.

31. A method of inhibiting hydroxylation of HIFα in a subject comprising identifying a subject in need thereof and administering to the subject, or contacting the subject with, an amount of at least one compound of Formula I-V, or a pharmaceutically acceptable salt, ester, amide, or prodrug thereof, sufficient to inhibit the hydroxylation of HIFα in the subject.

32. A method of modulating expression of HIF-regulated genes in a subject comprising identifying a subject in need thereof and administering to the subject, or contacting the subject with, an amount of at least one compound of Formula I-V, or a pharmaceutically acceptable salt, ester, amide, or prodrug thereof, sufficient to modulate expression of HIF-regulated genes in the subject.

33. A method of modulating HIF levels or HIF activity in a subject comprising identifying a subject in need thereof and administering to the subject, or contacting the subject with, an amount of at least one compound of Formula I-V, or a pharmaceutically acceptable salt, ester, amide, or prodrug thereof, sufficient to modulate HIF levels or HIF activity in the subject.

34. A method of treating a disorder in a subject where it is desired to modulate HIF activity or levels, the method comprising identifying a subject in need thereof and administering to the subject, or contacting the subject with, a therapeutically effective amount of at least one compound of Formula I-V, or a pharmaceutically acceptable salt, ester, amide, or prodrug thereof.

35. A method of treating a disorder in a subject comprising identifying a subject in need thereof and administering to the subject, or contacting the subject with, a therapeutically effective amount of at least one compound of Formula I-V, or a pharmaceutically acceptable salt, ester, amide, or prodrug thereof, wherein the disorder is selected from the group consisting of ischemic disorders, hypoxic disorders, anemic disorders (including, but not limited to, anemia associated with autoimmune diseases, rheumatoid arthritis, systemic lupus, chronic infections such as, without limitation, HCV, and HIV, inflammatory bowel disease, chemotherapy-induced, chronic heart disease, chronic kidney disease, chronic obstructive pulmonary disease (COPD), end stage renal disease, prematurity, hypothyroidism, malnutrition, blood disorders, including but not limited to, sickle cell anemia, and β-thalassemia, malignancies), stenocardia, neurological disorders, stroke, epilepsy, neurodegenerative disease, myocardial infarction, liver ischemia, renal ischemia, chronic kidney disease, peripheral vascular disorders, ulcers, burns, chronic wounds, pulmonary embolism, ischemic-reperfusion injury, ischemic-reperfusion injuries associated with surgeries and organ transplantations, respiratory distress syndrome, prevention of broncho-pulmonary dysplasia in pre-maturity, pulmonary hypertension, auto-immune diseases, side effects of diabetes, diabetic retinopathy, macular degeneration, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndrome, toxoplasmosis, trauma and post-laser complications, diseases associated with rubeosis, metabolic disorders, and proliferative vitreoretinopathy.

36. The method of claim 32, wherein the anemic disorder is selected from the group consisting of autoimmune disorders, chronic infections, inflammatory bowel disease, chronic heart disease, chronic kidney disease, chronic obstructive pulmonary disease (COPD), end stage renal disease, blood disorders, chemotherapy-induced, prematurity, hypothyroidism, malnutrition and malignancies.

37. The method of claim 36, wherein the blood disorder is sickle cell anemia or β-thalassemia.

38. The method of claim 36 wherein the chronic infection is HCV, or HIV.

39. The method of claim 36 wherein the autoimmune disorder is rheumatoid arthritis or systemic lupus.

40. A method of modulating the activity of a hydroxylase enzyme which modifies the alpha subunit of hypoxia inducible factor comprising contacting the enzyme with at least one compound of Formula I-V, or a pharmaceutically acceptable salt, ester, amide, or prodrug thereof.

41. A method of modulating levels of endogenous EPO in a subject comprising identifying a subject in need thereof and administering to the subject, or contacting the subject with, a therapeutically effective amount of at least one compound of Formula I-V, or a pharmaceutically acceptable salt, ester, amide, or prodrug thereof.

42. A method of regulating or modulating angiogenesis in a subject comprising identifying a subject in need thereof and administering to the subject, or contacting the subject with, a therapeutically effective amount of at least one compound of Formula I-V, or a pharmaceutically acceptable salt, ester, amide, or prodrug thereof.

43. A method for vascularizing ischemic tissue in a subject comprising identifying a subject in need thereof and administering to the subject, or contacting the subject with, a therapeutically effective amount of at least one compound of Formula I-V, or a pharmaceutically acceptable salt, ester, amide, or prodrug thereof.

44. A method for promoting the growth of skin graft replacements comprising identifying a subject in need thereof and administering to the subject, or contacting the subject with, a therapeutically effective amount of at least one compound of Formula I-V, or a pharmaceutically acceptable salt, ester, amide, or prodrug thereof.

45. A method for promoting tissue repair in the context of guided tissue regeneration (GTR) procedures comprising identifying a subject in need thereof and administering to the subject, or contacting the subject with, a therapeutically effective amount of at least one compound of Formula I-V, or a pharmaceutically acceptable salt, ester, amide, or prodrug thereof.

46. A method for treating anemia in a subject comprising identifying a subject in need thereof and administering to the subject, or contacting the subject with, a therapeutically effective amount of at least one compound of Formula I-V, or a pharmaceutically acceptable salt, ester, amide, or prodrug thereof.

47. A method for regulating anemia in a subject comprising identifying a subject in need thereof and administering to the subject, or contacting the subject with, a therapeutically effective amount of at least one compound of Formula I-V, or a pharmaceutically acceptable salt, ester, amide, or prodrug thereof.

48. A method for preventing anemia in a subject comprising identifying a subject in need thereof and administering to the subject, or contacting the subject with, a therapeutically effective amount of at least one compound of Formula I-V, or a pharmaceutically acceptable salt, ester, amide, or prodrug thereof.

49. A method of treating ischemia in a subject comprising identifying a subject in need thereof and administering to the subject, or contracting the subject with a therapeutically effective amount of at least one compound of Formula I-V, or a pharmaceutically acceptable salt, ester, amide, or prodrug thereof.

50. A method of treating a hypoxic-related disorder in a subject comprising identifying a subject in need thereof and administering to the subject, or contracting the subject with a therapeutically effective amount of at least one compound of Formula I-V, or pharmaceutically acceptable salt, ester, amide, or a prodrug thereof.

51. A method of treating inflammatory disorders in a subject comprising identifying a subject in need thereof and administering to the subject, or contracting the subject with, a therapeutically effective amount of at least one compound of Formula I-V, or a pharmaceutically acceptable salt, ester, amide, or prodrug thereof.
